(19) United States
(12) Patent Application Publication
(10) Pub. No.: US 2009/0205079 A1
(43) Pub. Date: Aug. 13, 2009

(54) ACETYL CoA CARBOXYLASE (ACCase) GENE FROM JATROPHA CURAS

(75) Inventors: Raja Krishna Kumar, Navi Mumbai (IN); Jain Devendra, Navi Mumbai (IN); Johnson Tangirala Sudhakar, Navi Mumbai (IN)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(73) Assignee: RELIANCE LIFE SCIENCES PVT. LTD., Navi Mumbai (IN)

(21) Appl. No.: 12/268,368

(22) Filed: Nov. 10, 2008

Related U.S. Application Data
(63) Continuation-in-part of application No. PCT/IN2007/000184, filed on May 8, 2007.

(30) Foreign Application Priority Data
May 9, 2006 (IN) 720/MUM/2006

(51) Int. Cl. A01H 1/00; C12N 9/00; C07H 21/00; A01H 5/00

(52) U.S. Cl. 800/281; 435/183; 536/23.2; 800/298; 800/278; 800/294; 800/292

(57) ABSTRACT

The present invention provides methods for increasing oil content in Jatropha Curcas L. plants. The invention further provides the cDNA and protein sequences of Jatropha acetyl CoA carboxylase (ACCase) and methods for cloning and expressing the Jatropha ACCase gene to produce transgenic plants with increased oil content.

### CROSS-REFERENCE TO RELATED APPLICATIONS

This application is a continuation-in-part of International Application Number PCT/IN2007/000184 filed May 8, 2007 designating the US and published as WO 2008/015692 A2, which claims the benefit of priority to Indian Application No. 720/MUM/2006, filed May 9, 2006, the contents of which is incorporated by reference herein in its entirety.

### TECHNICAL FIELD OF THE INVENTION

This invention relates to methods of increasing oil content of Jatropha curcas plants thereby enhancing the value of the plants. In particular, the invention is directed to a nucleic acid comprising the complete cDNA sequence and partial DNA sequence encoding Jatropha acetyl CoA carboxylase (ACCase). The invention also provides methods for cloning and expressing the Jatropha ACCase gene to produce transgenic Jatropha plants with increased oil content

### BACKGROUND OF THE INVENTION

Jatropha curcas is a plant of Latin American origin, widely spread throughout the arid and semi-arid tropical regions of the world. Jatropha is a large genus comprising over 170 species. The commonly occurring spp. in India are J. curcas, J. glandulifera, J. gossypifolia, J. multifida, J. nana, J. panduraefolia, J. villosa and J. podagrica. Jatropha curcas is used to produce the non-edible Jatropha oil for making candles and soap, and as a feedstock for the production of biodiesel. Jatropha has various medicinal uses especially in nutraceuticals, pharmaceutical, dermatological, and personal care products. The latex from Jatropha curcas has an anticancer properties associated with an alkaloid known as “jatrophine.”

Most of the Jatropha species are ornamental except for J. curcas and J. glandulifera which are oil yielding species. The seeds contain semi dry oil which has been found useful for medicinal and veterinary purposes. The oil content is 25-30% in the seeds and 50-60% in the kernel. The oil contains 21% saturated fatty acids and 79% unsaturated fatty acids. Jatropha oil are linolenic acid (C18:2) and oleic acid (C18:1) which together account for up to 80% of the oil composition. Palmitic acid (C16:0) and stearic acid (C18:0) are other fatty acids present in this oil. Thus, methods to introduce desirable traits in Jatropha species is needed. Seed yield and oil content are the most desirable traits in a species like Jatropha.

Acetyl-CoA carboxylase (ACCase, EC 6.4.1.2) has a very important regulatory role in controlling plant fatty acid biosynthesis and thereby affecting lipid biosynthesis. ACCase catalyzes the ATP-dependent carboxylation of acetyl-CoA to produce malonyl-CoA. This reaction occurs in two steps, carboxylation of a biotin prosthetic group using HCO₃ as a carboxyl donor, followed by a transfer of the carboxyl group from biotin to acetyl-CoA. The biotin carboxylase, carboxyl transferase, and biotin components of ACCase are each associated with different polypeptides in prokaryotes. Samols, D. et al., J. Biol. Chem. 263:6461-6464 (1988). In contrast, ACCase of non-plant eukaryotes is comprised of multimers of a single multi-functional polypeptide.

Both a prokaryotic type and an eukaryotic type ACCase have been found in plants. (Kannangara, C. G. et al., Arch. Biochem. Biophys. 152:83-91 (1972); Nikolau, B. J. et al., “The Biochemistry and Molecular Biology of Acetyl-CoA Carboxylase and Other Biotin Enzymes,” In N Murata, C Somerville, eds, Biochemistry and Molecular Biology of Membrane and Storage Lipids of Plants, American Society of Plant Physiologists, Rockville, Md. pp. 138-149 (1993) and Sasaki, Y. et al., J. Biol. Chem. 268:25118-25123 (1993)) (Harwood, J. L., Annu. Rev. Plant Physiol. Plant Mol. Biol. 39:101-138 (1988).

The malonyl-CoA produced by ACCase is involved in several biochemical reactions and pathways in plants, including fatty acid synthesis and elongation, flavonoid synthesis and malonation of the ethylene precursor aminocyclopropane-1-carboxylate, and malonation of amino acids and glycosides. (Harwood, J. L., Annu. Rev. Plant Physiol. Plant Mol. Biol. 39:101-138 (1988)) (Ebel, J. et al., Eur. J. Biochem. 75:201-209 (1977) Ebel, J. et al., Arch. Biochem. Biophys. 232:240-248 (1984)), (Liu, Y. et al., Planta 158:437-441 (1983); Kionka, C. et al., Planta 162:226-235 (1984)).

Malonyl-CoA is available in multiple subcellular locations because some of these reactions, such as fatty acid synthesis, occur in the plastid while others, such as flavonoid synthesis and fatty acid elongation, occur outside the plastid. For example, very long chain fatty acids are components of plasma membrane lipids (Cahoon, E. B. et al., Plant Physiol. 95:58-68 (1991)) and are also needed for synthesis of cuticular waxes to cover the surface of both aerial and underground tissues. Harwood, J. L., Annu. Rev. Plant Physiol. Plant Mol. Biol. 39:101-138 (1988). These very long chain fatty acids are synthesized outside the plastid by elongation of 16 or 18 carbon fatty acids exported from the plastid. Malonyl-CoA for the elongation reactions is present in the cytosol, and is presumably provided by a cytosolic ACCase.

Malonyl-CoA is available in greatly differing amounts with respect to time and tissue. For example, increased amounts of malonyl-CoA are needed for fatty acid synthesis in developing seeds of species, which store large quantities of triacylglycerols. Post-Beitenmiller, D. et al., “Regulation of Plant Lipid Biosynthesis: An Example of Developmental Regulation Superimposed on a Ubiquitous Pathway,” In DPS Verma, ed, Control of Plant Gene Expression, CRC press, Boca Raton, Fla. pp. 157-174 (1993). In floral tissue, malonyl-CoA is used in the chalcone synthase reaction for synthesis of the flavonoid pigments, which constitute up to 15% of the dry weight of this tissue. Goodwin, T. W. et al., “Introduction to Plant Biochemistry,” 2nd ed., Pergamon Press New York, p. 545 (1983). In some tissues, ACCase provides malonyl-CoA constitutively to produce fatty acids for membrane synthesis and maintenance, and for a short period to synthesize flavonoids during exposure to UV light (Ebel, J. et al., Eur J. Biochem. 75:201-209 (1977)) or during fungal pathogen attack. Ebel, J. et al., Arch. Biochem. Biophys. 232:240-248 (1984).

Various observations have led to the belief that ACCase is the rate-limiting enzyme for oilseed fatty acid synthesis. Analysis of substrate and product pool sizes has implicated ACCase in the light/dark regulation of fatty acid synthesis in spinach leaves and chloroplasts. Post-Beitenmiller, D. et al., J. Biol. Chem. 266:1858-1865 (1991) and Post-Beitenmiller, D. et al., Plant Physiol. 100:923-930 (1992). Further, ACCase activity increases in association with lipid deposition in developing seeds of oilseed crops. Simcox, P. D. et al., Canada J. Bot. 57:1008-1014 (1979); Turnham, E. et al., Biochem. J. 212:223-229 (1983); Charles et al., Phytochem. 25:55-59 (1986) and Deerburg, S. et al, Planta 180:440-444 (1990). Therefore, it is desirable to provide a gene encoding acetyl-CoA carboxylase (ACCase), which would control the carboxylation of acetyl-CoA to produce the fatty acid synthesizer, malonyl-CoA. To gain long term control of fatty acid synthesis and elongation in plants, seeds, cultures, cells and tissues of Jatropha curcas it is desirable to clone and obtain complete sequence of the ACCase gene, and later transform it into plant tissues through either Agrobacterium mediated or biolistic means under the stable plant promoter. This might provide genetically altered Jatropha plants with high oil content.

Roesler (1994) et al., Plant Physiol. 105: 611-617 have characterised an Arabidopsis gene that encodes cytosolic acetyl-CoA carboxylase (ACCase) isozyme. U.S. Pat. No. 6,723,895 discloses seeds of Soya Bean plants containing a recombinant nucleic acid construct comprising a cytosolic ACCase operably linked to a promoter. These seeds exhibit increased oil content compared to seeds produced by a corresponding plant not transformed with a nucleic acid encoding ACCase. However, these teachings are relatively specific to canola seed.

It also has been reported that ACCase increases the amount of malonyl-CoA available for synthesis of flavonoids, isoflavonoids, and other secondary metabolites. Conversely, decreasing expression of the ACCase gene may decrease the amount of malonyl-CoA present in a plant and increase the amount of acetyl-CoA. Thus, altering expression of the ACCase gene could alter the amount of acetyl-CoA or malonyl-CoA available for production of secondary plant products, many of which have value in plant protection against pathogens, or for medicinal or other uses. In view of this need in the art, no corresponding ACCase gene has been purified from Jatropha curcas until the present invention.

It has been observed in rapeseed, soybean, or other oilseed crops that overexpressing the ACCase gene would increase seed fatty acid synthesis resulting in increased oil content of rapeseed, soybean, or other oilseed crops. It has further been observed that decreasing seed fatty acid synthesis by decreasing ACCase gene expression results in “low-fat” seeds such as low-fat peanuts. Increasing seed fatty acid elongation by overexpressing the cytosolic ACCase gene is also useful in increasing the content of very long chain fatty acids such as erucic acid in the seed oil of rapeseed, Crambe, and other oilseed plants have been reported in Battey, J. F. et al., Trends in Biotech. 7:122-125 (1989). This is desirable because erucic acid and its derivatives can be used in making lubricants, plasticizers and nylons, and has other industrial uses as well. Although erucic acid has important industrial uses, it may not be healthy for human consumption in food products. Therefore, reducing fatty acid elongation, and thereby reducing erucic acid content, by decreasing the expression of cytosolic ACCase genes through anti-sense RNA methods, is also desirable. This may result in seed oil of rapeseed, mustard, Crambe and other oilseed plants that is suitable for human consumption because of the reduced content of erucic acid, eicosenoic acid and other very long chain fatty acids.

ACCase is also the target for herbicides of the aryloxyphenoxy propionate and cyclohexanedione families as reported in Burton, J. D. et al., Biochem. Biophys. Res. Commun. 148:1039-1044 (1987). The ACCase of some monocots such as corn is far more susceptible to these herbicides than is the ACCase of dicot species. Therefore, overexpression of the ACCase gene from the dicot Arabidopsis in plastids of susceptible species like corn, may result in herbicide resistance in the desired species. Herbicides would thus be useful in controlling monocot weeds in fields of the genetically engineered plant species. Studies have shown that, acetyl-CoA and malonyl-CoA are precursors of various plant secondary metabolites. Thus, increasing expression of the ACCase increases the amount of malonyl-CoA available for synthesis of flavonoids, isoflavonoids, plant fatty acid synthesis and other secondary metabolites.

A number of plant and animal cytosolic ACCases from organisms such as Arabidopsis thaliana (e.g., GenBank Accession No. L27074), Brassica napus (e.g., GenBank Accession No. X77576), Zea mays (e.g., GenBank Accession No. A25273) and Homo sapiens (e.g., GenBank Accession No. U19822). For example, a construct can contain a 35S cauliflower mosaic virus (CaMV) promoter, an alfalfa (i.e., Medicago saliva) cytosolic ACCase cDNA (e.g., GenBank Accession No. L25042), Saccharomyces cerivisiae (e.g., GenBank Accession No. M92156), Schizosaccharomyces pombe (e.g., GenBank Accession No. D78169), Ustilago maydis (e.g., GenBank Accession No. Z46886), Bos taurus (bovine) (e.g., GenBank Accession No. AJ132890), Rattus norvegicus (rat) (e.g., GenBank Accession No. AB004329), Ovis aries (sheep) (e.g., GenBank Accession No. X80045), Gallus gallus (chicken) (e.g., GenBank Accession No. J03541), Glycine max (soybean) (e.g., GenBank Accession No. L42814), Avena saliva (oat) (e.g., GenBank Accession No. AF072737), Triticum aestivum (wheat) (e.g., GenBank Accession No. U39321) and Phaseolus vulgaris (bean) (e.g., GenBank Accession No. AF007803), are known. (See U.S. Pat. No. 6,723,895 and WO 01/81604).

There is a need in the art for nucleic acids and proteins comprising the complete sequence of the Jatropha curcas ACCase gene, in order to gain long term control of fatty acid synthesis and elongation in plants, seeds, cultures, cells and tissues of Jatropha curcas. Thus, it is desirable to clone a nucleic acid comprising the complete sequence of the ACCase gene and use it to transform plant tissues under a stable plant promoter. This can provide genetically altered Jatropha plants with high oil content.

### SUMMARY OF THE INVENTION

It is an object of the present invention to provide complete cDNA sequence and genomic DNA sequence encoding Jatropha curcas ACCase enzyme.

It is an object of the present invention to provide an expression cassette comprising a gene coding for Jatropha curcas acetyl CoA carboxylase or a functional mutant thereof operably linked to a promoter functional in a plant cell.

It is an object of the present invention to provide methods for altering the oil content of plants by introducing and expressing Jatropha curcas acetyl CoA carboxylase gene in the plant cells.

It is also an object of the present invention to provide transgenic plant, which has increased oil content of at least 1.5-2 fold thus enhancing the commercial value of the plant.

The present invention relates to a nucleic acid sequence that can be used to increase and decrease the carboxylation of acetyl-CoA to produce malonyl-CoA in Jatropha curcas plants. In the present invention, the inventors disclose a cytosolic ACCase whose expression can control carboxylation of acetyl-CoA to produce malonyl-CoA. Keeping in mind that malonyl-CoA is required for fatty acid synthesis and elongation in plants and seeds, by overexpressing cytosolic ACCase, the inventors of the present invention have successfully developed a method that controls plant and seed fatty acid synthesis and elongation. Accordingly, the present invention contemplates the production of transgenic plants by expressing ACCase of Jatropha in those plants via constructs. Such a method further includes transforming the cytosolic ACCase into a plant cell, and growing the cell into a callus and then into a plant; or, alternatively, producing a transgenic plant directly through leaf disc transformation.

The present invention relates to isolating a sequence that may be used to generally increase and decrease the carboxylation of acetyl-CoA to produce malonyl-CoA in plants. In the present invention, the applicants have disclosed a partially sequenced cytosolic ACCase whose expression can control carboxylation of acetyl-CoA to produce malonyl-CoA. The invention in particular provides a complete cDNA sequence and genomic DNA sequences encoding Jatropha curcas acetyl CoA carboxylase.

The present invention further provides a method of increasing oil content of Jatropha curcas. The invention also provides methods for cloning and expressing the Jatropha ACCase gene to form transgenic plant with increased oil content. Accordingly, the present invention contemplates the production of a transgenic plant expressing Jatropha curcas acetyl CoA carboxylase (ACCase). ACCase can be introduced into the plants via an expression construct. This includes transforming a construct containing cytosolic ACCase into a plant cell and growing the cell into a callus and then into a plant. Alternatively, a transgenic plant can be produced directly through leaf disc transformation.

In one embodiment, the present invention provides an isolated and purified cDNA molecule that comprises a segment of cDNA encoding Jatropha ACCase gene. The cDNA molecule encoding a plant acetyl CoA carboxylase can encode an unaltered plant acetyl CoA carboxylase or an altered plant acetyl CoA carboxylase encoding an antisense cDNA sequence that is substantially complementary to a plant acetyl CoA carboxylase gene or to a portion thereof. A cDNA molecule of the present invention can also further comprise an amino terminal plant chloroplast transit peptide sequence operably linked to the Jatropha acetyl CoA carboxylase gene.

In another embodiment, the present invention provides methods of producing Jatropha plants with increased or altered oil content. The methods include introducing and expressing a plant ACCase gene in the plant cells. The methods further include the steps of introducing a chimeric cDNA molecule comprising a gene coding for a plant acetyl CoA carboxylase or an altered or a functional mutant thereof operably linked to a promoter functional in a plant cell into the cells of plant tissue and expressing the gene in an amount effective to alter the oil content of the plant cell.

In another embodiment, the present invention provides methods for an alteration in oil content which includes a change in total oil content over that normally present in that type of plant cell or a change in the type of oil present in the cell. An alteration in oil content in the plant cell, according to the method of the invention is achieved by at least two methods including: (1) an increase or decrease in expression of an altered plant acetyl CoA carboxylase gene; or (2) by introducing an altered or functional mutant plant acetyl CoA carboxylase gene.

In one embodiment, the method comprises the following steps: (i) isolation and identification of ACCase gene in Jatropha; (ii) formation of cDNA clones encoding ACCase; (iii) preparation of expression cassettes; (iv) introduction/transfer of expression cassettes in plant cells; (v) detection of the expression/activity of encoded gene in transgenic plant; and (vi) plant regeneration.

In one embodiment, the isolation and identification of gene coding for ACCase in Jatropha involves a genomic DNA or cDNA pool isolated and identified by using a degenerate primer strategy using standard methods as described by Sambrook et al., Molecular Cloning: A Laboratory Manual, 2nd ed., Cold Spring Harbor Laboratory Press (1989). The partial ACCase gene is incorporated herein in the detailed description. The presence of an isolated full-length copy of a plant ACCase gene is verified by partial sequence analysis, or by expression analysis of a plant acetyl CoA carboxylase enzyme.

In another embodiment of the present invention, the DNA fragments encoding portions of 5′, middle and 3′ ends are obtained which are used to construct expression cassettes containing ACCase gene. This method involves introducing a single or multiple copies of an expression cassette into plant cells. In a preferred embodiment the isolated unaltered ACCase gene is combined with or linked to a promoter that drives expression of the ACCase gene in a plant cell. An expression cassette includes the ACCase gene linked to a functional promoter. In another embodiment, the ACCase gene linked to a functional promoter is provided in an expression vector.

In one embodiment of the present invention, the promoters used for a high level of gene expression are inducible promoters, which are also known as strong promoters. In one embodiment the strong promoter used is an isolated sequence for heterologous genes expression, which facilitates detection and selection of transformed cells, while providing a high level of gene expression when desired. In a preferred embodiment of the present invention, the promoters specifically drive the over-expression of the ACCase gene or functional mutant thereof in a plant, thereby increasing acetyl-CoA and ultimately leading to increased malonyl-CoA levels in the plant. Such promoters include but not limited to 35 S cauliflower mosaic virus promoter, nopaline synthase (NOS) promoter and several other endosperm specific promoters such as Beta-phaseolin, napin, and ubiquitin.

In another embodiment of the present invention, the expression cassette can optionally contain other DNA sequences.

In yet another embodiment of the present invention, the expression cassette further comprises of a chloroplast transit peptide sequence operably linked between a promoter and a plant ACCase gene.

In another embodiment of the present invention, the expression cassette to be introduced into a plant cell contains plant transcriptional termination and polyadenylation signals and translational signals linked to the 3′ terminus of a plant ACCase gene.

In another embodiment of the present invention the DNA fragment coding for the transit peptide is chemically synthesized either wholly or in part from the known sequences of transit peptide.

In another embodiment of the present invention, the expression cassette comprising an ACCase gene is subcloned into a known expression vector. The method comprises introducing an expression vector into a host cell and detecting and/or quantitating expression of a plant ACCase gene. Suitable vectors include plasmids or other binary vectors. The expression vector can be introduced in prokaryotic or eukaryotic cells by protoplast transformation, Agrobacterium mediated transformation, electroporation, microprojectile or any technique known in the art. The selection of the transformed cells can be done by using markers encoded within the expression vector.

In one embodiment of the present invention, the detection of gene expression is done by PCR techniques or quantitatively detected by Western Blots. A change in the specific enzyme activity is detected by measuring the enzyme activity in transformed cells. The change in oil content is examined by standard methods.

In one embodiment of the present invention, the methods include regeneration of transgenic plants and seeds exhibiting a change in oil content or a change in the amount or the specific activity of the ACCase gene. Transgenic plants are produced by using standard techniques known in the art and the teachings herein. In preferred embodiments the present invention provides seeds with increased oil content of at least 1.5-2 fold thus enhancing the commercial value of the plant. In some embodiments the present invention provides seeds with increased oil content of at least 5 and up to 100% over non-transgenic seeds.

The present invention and other objects, features, and advantages of the present invention will become further apparent in the following Detailed Description of the invention and the accompanying figures and embodiments

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1 illustrates the amplification of 625 base pairs genomic fragment of ACCase gene from Jatropha curcas. One Kb markers (lane 1); genomic DNA of Jatropha curcas (Lane 5); genomic DNA of Arabidopsis and Medicago sativa (Lane 7 and 8).

FIG. 2A and FIG. 2B illustrate the nucleotide sequence of pGEM:J1 clone containing a 597 bp intermediate fragment of ACCase gene from Jatropha curcas. The primer binding sites (ME23 and ME25) are underlined.

FIG. 3A illustrates the nucleotide sequence of clone no. J-2 Forward (SEQ ID NO.2) and J-2 Reverse (SEQ ID NO. 3) containing 1.25 kb intermediate fragment of ACCase gene from Jatropha curcas. The 1.25 kb intermediate fragment of ACCase gene from Jatropha curcas is shown in FIG. 3B.

FIG. 4 illustrates the confirmation of the 1.25 kb amplified fragment of ACCase gene from Jatropha curcas.

FIG. 5 illustrates the nucleotide sequence of clone no. J-2 Forward (Seq ID No.2) and J-2 Reverse (SEQ ID NO. 3) containing 1.25 kb intermediate fragment of ACCase gene from Jatropha curcas. The sequence is characterized by: Length: 626 base pairs; Type: nucleic acid; Strandedness: double; Topology: Linear; Molecule type: DNA (genomic).

FIG. 6 illustrates the translated peptide sequence of Jatropha curcas clone J-1 acetyl-CoA carboxylase gene partial cds. (SEQ ID. NO: 4).

FIG. 7 illustrates the partial cds of Jatropha curcas clone J-2 acetyl-CoA carboxylase cds. (SEQ ID NO. 5). The sequence is characterized by: Length: 611 base pairs; Type: nucleic acid; Strandedness: single; Topology: Linear and Molecular type: mRNA.

FIG. 8 illustrates the translated peptide sequence of Jatropha curcas clone J-2 acetyl-CoA carboxylase gene, partial cds. (SEQ ID NO: 6). The sequence is characterized by: Length: 303 amino acids and Type: protein.

FIGS. 9A, B, C, and D illustrate partial cds of Jatropha curcas acetyl-CoA carboxylase mRNA (SEQ ID NO. 7). The sequence is characterized by: Length: 6634; Type: Nucleic acid; Strandedness: single; Topology: Linear; Molecule type: mRNA.

FIG. 10 illustrates the translated peptide sequence (SEQ ID. No. 8) of Jatropha curcase acetyl-CoA carboxylase mRNA partial cds. The sequence is characterized by: Length: 2211 and Type: protein.

FIGS. 11A-11F illustrate the Jatropha curcas genomic DNA sequence (SEQ ID NO: 9).

FIG. 12 illustrates construction of pCA-ME vector.

FIG. 13 illustrates cloning of J. curcas ACCase cDNA in two parts.

### DETAILED DESCRIPTION OF THE INVENTION

Without further elaboration, it is believed that one skilled in the art can, using the following description, utilize the present invention to its fullest extent. The following description is illustrative only, and not limiting of the remainder of the disclosure in any way whatsoever.

### Definitions

As used herein “gene transfer” refers to incorporation of exogenous DNA into an organism's cells usually, but not necessarily, by a vector.

As used herein, the term “transformed” refers to a cell, tissue, organ, or organism into which an exogenous polynucleotide molecule, such as a construct, has been introduced. Preferably, the introduced polynucleotide is integrated into the genomic DNA of the recipient cell, tissue, organ, or organism such that the introduced polynucleotide molecule is inherited by subsequent progeny.

A “genetically modified plant” is a plant that has undergone genetic modification through a technique whereby one or more individual genes have been copied and transferred to the plant to alter its genetic make up and, thus, incorporate or delete specific characteristics into or from the plant.

A “transgenic plant” refers to a genetically modified plant with a new gene (transgene) that may impart a new function. For example, transgenic plants are plants that have been genetically engineered using recombinant DNA technology to make plants with new characteristics (e.g., increased oil content). Transgenic plants are produced by adding one or more genes to a plant genome, often via transformation.

A “transgenic” or “transformed” cell or organism includes progeny of the cell or organism and progeny produced from a breeding program employing such a transgenic plant as a parent in a cross and exhibiting an altered phenotype resulting from the presence of an exogenous polynucleotide molecule.

As used herein “Agrobacterium mediated transformation” is the use of Agrobacterium to transfer DNA to plant cells harnessed for the purposes of plant genetic engineering

As used herein “increased” or “altered” or “high” “oil content” Jatropha curcas plant is one having seeds with increased oil content. For example, Jatropha curcas having seeds above 30% oil content is considered to be a “high oil content” plant. An increased oil content plant has seeds containing at least 5, 10, 15, 20, 25, 30, 40, 50, 60, 70, 80 or 100% more oil as compared to a plant having unaltered expression of Jatropha curcas ACCase.

In accordance with the embodiments, the present invention provides for the isolation and identification of the ACCase gene in Jatropha plant. The gene encoding plant ACCase was identified and isolated from a gDNA or cDNA pool of Jatropha curcas L. using a degenerate primer strategy. Its partial sequence was obtained by standard methods, as described by Sambrook et al., (1989) which is incorporated herein by way of reference in the present invention. The sequence homology was compared with other known acetyl CoA carboxylase. The DNA fragments encoding portions of the 5′, middle and 3′ ends obtained were used to construct an expression cassette containing the ACCase gene. Presence of an isolated full-length copy of a plant ACCase gene was verified by partial sequence analysis, or by expression of a plant acetyl CoA carboxylase enzyme.

A nucleic acid according to the invention comprises a multi-functional cytosolic Jatropha curcas acetyl coA-carboxylase (ACCase) coding sequence operably linked to a promoter. The genomic DNA, cDNA or partial DNA sequence is combined with a suitable promoter to form a recombinant expression cassette. This expression cassette is then transferred/introduced and expressed in a plant cells. The plant cells are then detected for enzyme activity or gene expression and then further regeneration for plants with high oil content.

After the plant ACCase gene is obtained and amplified, it can then combined with a suitable promoter functional in a plant cell to form an expression cassette. As used herein, “promoter” refers to nucleic acid sequences that, when operably linked to an ACCase coding sequence, direct transcription of the coding sequence such that it's gene product can be produced. Suitable promoters are known in the art (Weising et al., Ann Rev. Genetics 22:421-478 (1988)).

The following representative promoters are suitable for use in the invention described herein: regulatory sequences from fatty acid desaturase genes (e.g. Brassica fad2D or fad2F, see WO 00/07430); alcohol dehydrogenase promoter from corn; light inducible promoters such as the ribulose bisphosphate carboxylase (Rubisco) small subunit gene promoters from a variety of species; major chlorophyll a/b binding protein gene promoters; the 19S promoter of cauliflower mosaic virus (CaMV); a seed-specific promoter such as a napin or cruciferin seed-specific promoter; as well as synthetic or other natural promoters.

An expression cassette of the invention comprises a gene encoding a Jatropha curcas acetyl CoA carboxylase or functional mutant thereof operably linked to a promoter functional in a plant cell. A nucleic acid encoding a cytosolic ACCase may or may not contain introns within the coding sequence. The gene codes for a J. curcas ACCase that can alter the oil content of the plant cell. It should be appreciated that many different nucleic acids can encode a J. curcas ACCase polypeptide sequence. Due to degeneracy of the genetic code many amino acids are coded for by more than one nucleotide codon. Amino acid substitutions can be made within polypeptide sequences without affecting the function of the polypeptide. Conservative amino acid substitutions or substitutions of similar amino acids often are tolerated without affecting polypeptide function. Dayhoff et al. (1978) in Atlas of Protein Sequence and Structure, Vol. 5, Suppl. 3, pp. 345-352. An expression cassette of the invention also includes an antisense DNA sequence that is complementary to a Jatropha curcas ACCase gene or a portion thereof operably linked to a promoter. The promoter is selected from constitutive or tissue specific promoters such as endosperm specific promoters. An expression cassette with antisense DNA of the a Jatropha curcas ACCase gene can be used to decrease the expression of the a Jatropha curcas ACCase in a plant cell.

Most genes have regions of DNA sequences that are known as promoters, which regulate gene expression. Promoter regions are typically found in the flanking DNA sequence upstream from the coding sequence in both prokaryotic and eukaryotic cells. A promoter sequence provides for regulation of transcription of the downstream gene sequence and typically includes from about 50 to about 2000 nucleotide base pairs. Promoter sequences also contain regulatory sequences such as enhancer sequences that can influence the level of gene expression. Some isolated promoter sequences can provide for gene expression of heterologous genes, that is a gene different from the native or homologous gene. Promoter sequences are also known to be strong or weak or inducible. A strong promoter provides for a high level of gene expression, whereas a weak promoter provides for a very low level of gene expression. An inducible promoter is a promoter that provides for turning on and off of gene expression in response to an exogenously added agent or to an environmental of developmental stimulus. Promoters can also provide for tissue specific or developmental regulation. An isolated promoter sequence that is a strong promoter for heterologous genes is advantageous because it provides for a sufficient level of gene expression to allow for easy detection and selection of transformed cells and provides for a high level of gene expression when desired. Specific promoters functional in plant cells include the 35S cauliflower mosaic virus promoter, nopaline synthase (NOS) promoter and the like. A preferred promoter for expression is the 35S cauliflower mosaic virus promoter and several endosperm specific promoters such β-phaseolin, napin and ubiquitin, however the invention is not limited to such promoters.

An ACCase gene can be combined with the promoter by standard methods as described in Sambrook supra. Briefly, a plasmid containing a promoter such as the 35S cauliflower mosaic virus promoter can be constructed as described in Jefferson, (Plant Molecular Biology Reporte 5: 387 (1987)) or obtained from Clontech Lab, Mountain View, Calif. (e.g. pBI121 or pBI221). Typically these plasmids are constructed to provide for multiple cloning sites having specificity for different restriction enzymes downstream from the promoter. A gene for plant ACCase can be subcloned downstream from the promoter using restriction enzymes to ensure that the gene is inserted in proper orientation with respect to the promoter so that the gene can be expressed. In a preferred version, a Jatropha ACCase is operably linked to a 35S CaMV, or β-phaseolin or napin or ubiquitin promoter in a plasmid such as pBI121 or pBI221. Once a plant ACCase gene is operably linked to a promoter and the plasmid, the expression cassette so formed can be further subcloned into other plasmids or vectors.

The expression cassette can also optionally contain other DNA sequences. The expression cassette further can comprise a chloroplast transit peptide sequence operably linked to a promoter and adjacent a plant ACCase gene. If the expression cassette is to be introduced into a plant cell, the expression cassette can also contain plant transcriptional termination and polyadenylation signals and translational signals linked to the 3′ terminus of a plant ACCase gene. As one site of action for biosynthetic pathways involving plant ACCase is the chloroplast, an expression cassette can be combined with a DNA sequence coding for a chloroplast transit peptide, if necessary. A chloroplast transit peptide is typically 40 to 70 amino acids in length and functions post translationally to direct the protein to the chloroplast. The transit peptide is cleaved either during or just after import into the chloroplast to yield the mature protein. The complete copy of a gene encoding a plant ACCase may contain a chloroplast transit peptide sequence. In that case, it may not be necessary to combine an exogenously contained chloroplast transit peptide sequence into the expression cassette.

Transit peptide sequences are the small subunit of ribulose biphosphate carboxylase, ferridoxin, chlorophyll a/b binding protein, and so on. Alternatively, the DNA fragment coding for the transit peptide may be chemically synthesized either wholly or in part from the known sequences of transit peptide. Regardless of source of transit peptide, it includes a translation initiation codon and an amino acid sequence that is recognised by and will function properly in chloroplasts of the host plant. The amino acid sequence at the junction between the transit peptide and the plant ACCase is an essentially responsible for cleaving, to yield a mature protein (e.g., the enzyme).

The invention also provides for a method of producing plant ACCase in a host cell. The methods include the steps of introducing an expression cassette comprising a gene encoding a plant ACCase. An expression cassette can include a promoter that is functional in either a eukaryotic or a prokaryotic cell. Preferably, the expression cassette is introduced into a prokaryotic cell such as E. coli that is routinely used for production of recombinantly produced proteins. An expression cassette can be introduced into either monocots or dicots by standard methods including protoplast transformation, Agrobacterium mediated transformation, microprojectile bombardment, electroporation and the like. Techniques are known for the introduction of DNA into dicots as well as monocots, as are the techniques for culturing such tissues and regenerating plants. If cell or tissue cultures are used as the recipient tissue for transformation, plants can be regenerated from transformed cultures by techniques known to those skilled in the art. Suitable dicots include plants such as alfalfa, soybean, rapeseed (high erucic and canola), and sunflower. Monocots that have been successfully transformed and regenerated in the art include wheat, corn, rye, rice, sorghum and asparagus (see, U.S. Pat. Nos. 5,484,956 and 5,550,318).

Preferred species for generating transgenic plants of the present invention include, without limitation, oil-producing species, such as soybean (Glycine max), rapeseed (e.g., Brassica napus, B. rapa and B. juncea) (both Spring and Winter maturing types within each species), sunflower (Helianthus annus), castor bean (Ricinus communis), safflower (Carthamus tinctorius), palm (e.g., Elaeis guineensis), coconut (e.g., Cocos nucifera), meadowfoam (e.g., Limnanthes alba alba and L. douglasii), cottonseed (e.g. Gossypium hirsutum), olive (e.g., Olea europaea), peanut (e.g., Arachis hypogaea), flax (e.g., Linum usitatissimum), sesame (e.g., Sesamum indicum) and crambe (e.g., Crambe abyssinica or C. hispanica). Accordingly, suitable families include, but are not limited to, Solanaceae, Leguminaceae, Brassicaceae and Asteraccae. A transgenic plant of the invention typically is a member of a plant variety within the families or species mentioned above. Transformed tissues or cells can be selected for the presence of a selectable marker gene.

A method for screening for expression or overexpression of a plant ACCase gene is also provided by the invention. Once formed, an expression cassette comprising an ACCase gene can be subcloned into a known expression vector. The screening method includes the steps of introducing an expression vector into a host cell and detecting and/or quantitating expression of a plant ACCase gene.

Transient expression of a plant ACCase gene can be detected and quantitated in the transformed cells. Gene expression can be quantitated by a quantitative Western blot using antibodies specific for the cloned ACCase or by detecting an increased specific activity of the enzyme. Expression cassettes providing for overexpression of plant ACCase can be then used to transform monocots /and or dicot tissues to regenerate transformed plant and seeds. Transgenic plants are created by introducing an ACCase nucleic acid construct into a plant cell and growing the plant cell into a plant. Such plants contain and express the ACCase nucleic acid construct. Suitable techniques for introducing nucleic acids into plant cells to create such plants include, without limitation, Agrobacterium-mediated transformation, viral vector-mediated transformation, electroporation and particle gun transformation. Illustrative examples of transformation techniques are disclosed in U.S. Pat. No. 5,204,253, (biolistic transformations), U.S. Pat. No. 6,051,756 (biolistic transformation of Brassica) and U.S. Pat. No. 5,188,958 (Agrobacterium transformation). Transformation methods utilizing the Ti and Ri plasmids of Agrobacterium spp. typically use binary-type vectors (e.g., ptet1, pBin19) (Walkerpeach et al., in Plant Molecular Biology Manual, Gelvin & Schilperoort, eds., Kluwer Dordrecht, C1: 1-19 (1994)).

The invention also provides a method of altering the oil content in a plant. The method includes the steps of introducing an expression cassette comprising a gene coding for plant ACCase operably linked to a promoter functional in a plant cell, into the cells of plant tissue and expressing the gene in an amount effective to alter the oil content of the plant cell. An alteration in the oil content of a plant cell can include a change in the total oil content over that normally present in the plant cell. Expression of the gene in an amount effective to alter the oil content of the gene depends on whether the gene codes for an unaltered ACCase or a mutant or altered form of the gene. Expression of an unaltered plant ACCase in an effective amount is that amount may provide a change in the oil content of the cell at least about 1.2 to 2 fold over that normally present in that plant cell, and preferably increases the amount of ACCase about 1.05- to 20-fold over that amount of the enzyme normally present in the plant cell. An altered form of the enzyme can be expressed at levels comparable to that of the native enzyme or less if the altered form of the enzyme has higher specific activity.

A DNA sequence corresponding to J. curcas ACCase genomic DNA was deposited GenBank under Accession No. 1053703 (Jan. 11, 2008). The DNA sequence corresponding to full-length mRNA sequence inclusive of 5′ and 3′ UTR are available at GenBank accession number: GI:157427567 (under protection mode)

TABLE 1
DNA sequence corresponding to full-length mRNA sequence
of J. curcas ACCase (7715 bases). SEQ ID NO: 1

| | |
|---|---|
| 1 | gagtttggtg tttgaagtat cgggatagta ttgtttttag cagcatagtg atgggaagtt |
| 61 | tctatgctgt tctttttatg ggtggttttt aaatttttat tgatcaaatt ttagttatgg |
| 121 | atttgggaat ctaattgtgc tcttttgtgg gggcgaatga ctattttatg ttattgggca |
| 181 | ctttttcatc gttatcatca atatcctagg attctgcacc tgagatactt tgacttttaa |
| 241 | actgtgtttg gtttcttata acttaagtgg aatatactaa tgaattgggg ttcggtttta |
| 301 | gtgatttaac tctgattatc aaacaactac acgatcaagt ctagagttta tagtcccagg |
| 361 | aagtaaagaa gtctacatca attccttatt tgcaaataaa gttgttaaat tgttctgtca |
| 421 | acttttgctc acgaactatg aacaaatgat attactaagg aaggcctacc cgaaaagtaa |
| 481 | aactgtattt aagtataaga aaacgattga cgttgagaat gatttaattt gaagnctcaa |
| 541 | ttttggtaac aaaagtccta tctatgcagc atgttggaag cacaaaggag accaccggaa |
| 601 | ccggtgggtg ttgctcgtgt aatggttaca taaatggggt agtttcaatg agaagtcctg |
| 661 | ctacaatatc tgaagtggat gaattctgcc atgctcttgg agggaatagt ccaattcata |
| 721 | gtattttaat agcaaacaat ggaatggcag ctgtcaagtt tatgcgtagt attagaacat |
| 781 | gggcttatga aacatttggc aatgagaagg caatcttgtt ggtggccatg gcaactccgg |
| 841 | aagacatgaa aatcaatgca gagcatatta gaattgctga tcaatttgta gaagttcctg |
| 901 | gtgggacaaa caataataac tatgccaatg tgcagctgat cttagagatg gcagaaggaa |
| 961 | ctcgtgttga tgccgtttgg cctggttggg gccatgcatc tgagaaccct gagctgccag |
| 1021 | atgcactgag tgcaaaaggt atcgtatttc ttgggccccc agctacatct atggctgcac |
| 1081 | tgggtgataa aatcggctca tctttgattg ctcaagcagc agatgtccct actcttccat |
| 1141 | ggagtggctc tcatgtgaaa attcctccag aaagttgttt gattgccatc ccagatgagg |
| 1201 | tatacagaga agcatgtgta tatacaacag aggaagcgat tgcaagttgt caagttgttg |
| 1261 | gataccctgc aatgatcaag gcatcatggg gtggtggtgg taaaggcata agaaaggttc |
| 1321 | ataatgatga tgaagtcagg gcattgttca agcaagttca aggtgaagtt ccaggatcac |
| 1381 | ccatatttat aatgaaggtt gcttcccaga gtcgacattt ggaagtccag ttactctgtg |
| 1441 | atcagcatgg gaatgtagct gctttgcaca gccgtgattg cagtgttcag aggcggcacc |
| 1501 | aaaagataat tgaggagggt ccaattactg ttgcgcctct ggagacagtc aaaaagctag |
| 1561 | aacaagcagc tcgaaggtta gcgaaaagtg tgaattatgt tggagcagct actgttgagt |
| 1621 | atttgtacag tatggaaact ggagaatact actttttaga actcaatcct cggttacagg |
| 1681 | tggagcaccc agtgactgag tggattgctg aagtaaattt gccagctgcc caggtagctg |
| 1741 | ttgggatggg aattcctctc tggcaaattc ctgagataag gcgattttat ggagtggaaa |
| 1801 | atggtggagg atatgatgct tggaggaaaa cttcagtggt tgctactcct tttgattttg |
| 1861 | acaaggctga gtctactagg ccaaaaggcc attgtgtggc tgtgcgtgtg acaagtgagg |
| 1921 | atccagatga tggttttaag cctacaagtg gaaaagtaca ggagctaagt tttaaaagca |
| 1981 | agccaaatgt gtgggcttat ttctctgtta agtctggtgg aggcattcat gaattttcag |
| 2041 | attctcaatt tggtcatgtt tttgcgtttg gagaatccag agctttggct atagcaaata |
| 2101 | tggtccttgg gctgaaagaa attcaaattc gaggagaaat tcggactaat gttgactact |
| 2161 | caattgatct tttacacgct tctgactata gggacaacaa aatccacaca ggttggttgg |
| 2221 | acagtagaat tgcaatgcgg gttagagcaa aaaggccccc ttggtacctc tctgttgttg |
| 2281 | gaggggcttt atacaaagca tctgctagca gtgcagctat ggtttcagat tatgttggtt |
| 2341 | accttgaaaa ggggcaaatc cctcctaagc acatatcact tgttaactct caagtttcat |
| 2401 | tgaacattga aggaagcaaa tacgtgataa acatggttag aggggggcca ggaagctata |
| 2461 | gattgagaat gaatgaatca gagattgaag eagagataca tactttacgt gatggaggtt |
| 2521 | tattgatgca gttggatgga aacagtcatg tgatatatgc agaagaagaa gcagctggaa |
| 2581 | ctcgtcttct tattgatgga aggacttgct tgctgcagaa tgatcacgat ccttcaaagt |
| 2641 | tagtggcaga aacgccatgc aagctgctga ggtttttggt tttggatggt agtcatattg |
| 2701 | aagctgatac tccatatgcg gaggttgagg tcatgaagat gtgcatgcct ctcctttcac |
| 2761 | ctgcttctgg agttcttcag tttaaaatgt ctgaaggtca agcaatgcag gctggtgagc |
| 2821 | ttatagcacg gcttgaactt gatgatcctt cggctgtacg aaagcctgaa ctttttcatg |
| 2881 | ggagcttccc aatactgggg ccaccaactg ctatttctgg taaagttcat cagagatgtg |
| 2941 | ctgcaagtct gaatgcagct tgcatgattc ttgctggcta tgaacacaat attgatgaag |
| 3001 | tagtacaaaa cttgctaaac tgtctagaca gtcctgaact acctttcctt cagtggcaag |
| 3061 | agtgcttgtc tgttctggca actcgccttc ccaaagatct tagaaatgag ttggaatcaa |
| 3121 | aatacagggg gtttgaaggg atttcgagct cccagaatgt tgacttccct gccaaattgt |
| 3181 | taaggggtgt tcttgaggcc catctatcct cctgtcctga aaaagaaaaa ggtgcacaag |
| 3241 | aaaggcttgt tgaacctttg atgagtcttg taaagtctta tgagggagga cgggagagtc |
| 3301 | atgcccgcgt cattgttcag tcactttttg acgagtattt atctgttgaa gaattgttca |
| 3361 | gagataacat ccaggctgat gtgattgaac gtcttagact ccaatacaag aaagatctgt |
| 3421 | tgaaggttgt tgacattgtc ctttctcatc agggtgtgag gagtaaaaat aagctgatat |
| 3481 | tgcggcttat ggaacaattg gtttatccta accctgctgc atatagggat aaactgatcc |
| 3541 | gcttctctca acttaaccat acaagttatt ctgagttggc actgaaggca agtcaactgc |
| 3601 | tagaacaaac caaactaagt gaacttcgtt ccatcattgc tagaagcctc tctgaattgg |
| 3661 | agatgtttac tgaggatggt gaaaatatgg atactcctaa gaggaaaagt gccattaatg |
| 3721 | aacgaatgga ggatctagtg agcgctcctt tggctgttga ggatgctctt gtggggctgt |
| 3781 | ttgatcacag tgatcacact cttcagaggc gggtggtgga aacctatgtt cgaaggctat |
| 3841 | accagccata tcttgtaaag gagagtgtca ggatgcagtg gcatagatct ggtctgattg |
| 3901 | cttcatggga gttcttggaa gaacatattg gaagaaagaa tggctatgaa gatcaaatgt |
| 3961 | ctgatgaacc agtaatggag aaacactgtg acaggaaatg gggagccatg gttattatca |
| 4021 | aatctctaca gtttttacct gcaattatta gtgctgcact aagagaaacg acccacaatc |
| 4081 | ttcatgaagc cattccaaat agatctacag aactagataa ctatggtaat atgatgcata |
| 4141 | ttgctttggt gggcatcaac aaccagatga gtctacttca ggatagtggt gatgaggatc |
| 4201 | aggctcaaga gagaattaaa aagttagcaa aaattcttaa agaacaagaa gtaggctcca |
| 4261 | gtttgcgcac cgcaggtgtt gaagttatta gctgcatcat acaaagggat gaaggaaggg |
| 4321 | cccctatgag acactccttt cactggtcag aagaaaagct ctactatgag gaagaacctc |
| 4381 | tattgcgaca tctagaacct ccactgtcca tctatctaga attggataaa cttaaaagtt |
| 4441 | atgggaacat acagtacact ccatcacggg acagacagtg gcacttgtac actgttgtag |
| 4501 | acaagccagt gtcaatccag aggatgtttc ttagaaccct tgtgaggcaa cctacaacaa |
| 4561 | atgaagtgtt caccgcatgt caaggactgg gcatggaagc acctcaagca caatggacta |
| 4621 | tgtcctttac ttcaagaagc attttgaggt ccttagtggc tgcgatggag gagttggaac |
| 4681 | ttaatatgca taatgctact gtcaaatctg accatgctca tatgtatctc tgtattttgc |
| 4741 | gggagcaaca aatagatgat cttgtgccat accccaagag agttgatatt gaggctggcc |
| 4801 | aggaagaagt tgcaattggc cgaatcttgg aagaactggc tagggaaata catgcatccg |
| 4861 | ttggtgtgaa aatgcatagg ttaaatgttt gtgaatggga agtgaagctc tggatgacat |
| 4921 | catgtggaca ggcaaatggt gcttggcgag ttgttatcac taatgtaact ggtcacacct |
| 4981 | gtgctgtaca tacataccgg gaactagagg atgccagcaa acatggagtg gtgtaccatt |
| 5041 | cagtctctgt acagggtcct ctgcatggtg tattggtaaa tgcagtttat cagtccttgg |
| 5101 | gagttcttga tcgaaaacgt cttttggcaa ggagaagcaa caccacatac tgctacgatt |
| 5161 | ttccactggc atttgagaca gccttggaac aaatatgggc atcccagttt actggaactg |
| 5221 | gaaaactgaa gtgtaatgtt cttgtcaaag ccacagagct tgtattttct gatcagaaag |
| 5281 | gcagctgggg tactcctctt gttcctgtgg atcgcccagc tgggctcaat gacattggca |
| 5341 | tgatagcatg gaccatggaa ttgtctaccc ctgagtttcc ttctggaagg acaattttga |
| 5401 | tagtagcaaa tgatgtcacc ttcaaagctg ggtcttttgg cccaagagag gatgcattct |
| 5461 | tctatgctgt aaccgatctt gcttgcacaa aaaagcttcc attaatttat ttggcagcaa |
| 5521 | attctggtgc ccgaattggg gttgccgagg aagtgaaatc ctgttttaaa gttggttggt |
| 5581 | cagatgaaac atcccctgag ggtggttttc aatatgtata tttgagtcct gaagattaca |
| 5641 | ctcacattgc atcatctgtc atagcacatg agttgaagct atctaatgga gaaaccagat |
| 5701 | gggtgataga tgccattgtt ggaaaggagg atggcttggg ggtagagaac ttatctggaa |
| 5761 | gtggggccat tgctagtgca tattctaggg catacaaaga aacttttacc ttaacatatg |
| 5821 | tcacaggtag aacagtggga attggagctt acctagctcg gcttgggatg cgatgcatgc |
| 5881 | aaagggttga tcagcccatt attttgactg gtttctctgc attgaacaaa cttcttggtc |
| 5941 | gtgaggtgta cagctctcac atccaacttg gtggccccaa agttatggca accaatggag |
| 6001 | tagttcatct tactgtctca gatgatctag aaggtgtatc tgctatcttg aactggctaa |
| 6061 | gttgtatccc tccttgtatt ggtggcacac ttccaatttt aggtccttcg gatcctactg |
| 6121 | aaaggcctgt ggagtatttc ccagaaaact catgtgatcc acgtgctgct atttctggtt |
| 6181 | ctttggatgg taatgggaag tggcttgggg gcatttttga caagaatagt tttgttgaga |
| 6241 | cactggaagg ctgggcaagg acagttgtga caggaagggc aaagctcgga ggaatccctg |
| 6301 | ttggagtaat agctgttgaa actcaaactg tgatgcaggt gattcctgct gacccaggac |
| 6361 | agctcgattc tcatgagagg gttgttcctc aggctggcca agtatggttt ccagattctg |
| 6421 | caaccaaaac agctcaagct atattggatt tcaacagaga agaacttcca cttttcattc |
| 6481 | ttgcatattg gaggggcttt tcaggtggac aaagggatct ttttgaaggt atcctccagg |
| 6541 | caggttcaac aatagttgag aatcttagga catacaacca acctgttttt gtatacatcc |
| 6601 | ccatgatggg tgaacttcgt ggtggggcat gggttgtggt ggacagtcag atcaattctg |
| 6661 | accatataga aatgtatgct gataggacag ccaaaggtaa tgtccttgag ccagaaggca |
| 6721 | taattgagat caaatttaga acaaaagagc tgcttgagtc catgggtagg cttgataaac |
| 6781 | agttgatcac attgaaggca aaacttcaag aagctaggaa tagctggaac tttgggatgg |
| 6841 | ttgaagactt acaacagcag ataaaatctc gtgaaaagca acttttgccc atatacactc |
| 6901 | aaatagccac cagatttgcg gagcttcatg attcttccct aaggatggct gcaaaggggg |
| 6961 | tgatcagaga aattgtagac tgggataaat cccgtgctta cttctataaa aggctacgta |
| 7021 | ggagaatcgc tgagggttca ctgatcaaga ctgtgaaaga tgcagctggt gaccagttgt |
| 7081 | cccataaatc tgcaatggac ttgatcaaaa actggttttt agattctgat attgcaagag |
| 7141 | gcaaagaaga tgcttggggg aatgatgaag ctttctttgc atggaaggat gatcaaggga |
| 7201 | aatatgaaga aaaactacaa gagctacggg ttcagaaagt gttggtacaa ctgacaaaca |
| 7261 | ttggtgactc catgtcagat ttgaaagctc tacctcaagg tcttgctgct cttctaagaa |
| 7321 | aggtggagcc atcgagccga gggcaaataa ttgaagagct tcgaaaggtc atcagttgat |
| 7381 | ttggtatgtc ctttacgagc gaatattcat gctcatactt aggtaacaga tattttcaag |
| 7441 | tgagaaaaag aaatgtattt acaatgctat ttgccaaccc tatatgcaat tgtaatttat |
| 7501 | cagccaagag gaaaacctca ctgtaaattg gagaaggttc tccaccgatc agttttaatg |
| 7561 | cttcagtgta aatttagctt taatcttggg ataaactagg agtagattga tattgttaag |
| 7621 | agtggaaact ggccagcatt ggcagcctat gccatccatg gctgttcctt ggcttgttta |
| 7681 | gttattattt ttgaaataaa aaaaaaaaaa aaaaa |

TABLE 2
Full-length translated protein sequence of
J. curcas ACCase (2271 amino acids). SEQ ID NO: 2

| | |
|---|---|
| 1 | MQHVGSTKET TGTGGCCSCN GYINGVVSMR SPATISEVDE FCHALGGNSP IHSILIANNG |
| 61 | MAAVKFMRSI RTWAYETFGN EKAILLVAMA TPEDMKINAE HIRIADQFVE VPGGTNNNNY |
| 121 | ANVQLILEMA EGTRVDAVWP GWGHASENPE LPDALSAKGI VFLGPPATSM AALGDKIGSS |
| 181 | LIAQAADVPT LPWSGSHVKI PPESCLIAIP DEVYREACVY TTEEAIASCQ VVGYPAMIKA |
| 241 | SWGGGGKGIR KVHNDDEVRA LFKQVQGEVP GSPIFIMKVA SQSRHLEVQL LCDQHGNVAA |
| 301 | LHSRDCSVQR RHQKIIEEGP ITVAPLETVK KLEQAARRLA KSVNYVGAAT VEYLYSMETG |
| 361 | EYYFLELNPR LQVEHPVTEW IAEVNLPAAQ VAVGMGIPLW QIPEIRRFYG VENGGGYDAW |
| 421 | RKTSVVATPF DFDKAESTRP KGHCVAVRVT SEDPDDGFKP TSGKVQELSF KSKPNVWAYF |
| 481 | SVKSGGGIHE FSDSQFGHVF AFGESRALAI ANMVLGLKEI QIRGEIRTNV DYSIDLLHAS |
| 541 | DYRDNKIHTG WLDSRIAMRV RAKRPPWYLS VVGGALYKAS ASSAAMVSDY VGYLEKGQIP |
| 601 | PKHISLVNSQ VSLNIEGSKY VINMVRGGPG SYRLRMNESE IEAEIHTLRD GGLLMQLDGN |
| 661 | SHVIYAEEEA AGTRLLIDGR TCLLQNDHDP SKLVAETPCK LLRFLVLDGS HIEADTPYAE |
| 721 | VEVMKMCMPL LSPASGVLQF KMSEGQAMQA GELIARLELD DPSAVRKPEL FHGSFPILGP |
| 781 | PTAISGKVHQ RCAASLNAAC MILAGYEHNI DEVVQNLLNC LDSPELPFLQ WQECLSVLAT |
| 841 | RLPKDLRNEL ESKYRGFEGI SSSQNVDFPA KLLRGVLEAH LSSCPEKEKG AQERLVEPLM |
| 901 | SLVKSYEGGR ESHARVIVQS LFDEYLSVEE LFRDNIQADV IERLRLQYKK DLLKVVDIVL |
| 961 | SHQGVRSKNK LILRLMEQLV YPNPAAYRDK LIRFSQLNHT SYSELALKAS QLLEQTKLSE |
| 1021 | LRSIIARSLS ELEMFTEDGE NMDTPKRKSA INERMEDLVS APLAVEDALV GLFDHSDHTL |
| 1081 | QRRVVETYVR RLYQPYLVKE SVRMQWHRSG LIASWEFLEE HIGRKNGYED QMSDEPVMEK |
| 1141 | HCDRKWGANV IIKSLQFLPA IISAALRETT HNLHEAIPNR STELDNYGNM MHIALVGINN |
| 1201 | QMSLLQDSGD EDQAQERIKK LAKILKEQEV GSSLRTAGVE VISCIIQRDE GRAPMRHSFH |
| 1261 | WSEEKLYYEE EPLLRHLEPP LSIYLELDKL KSYGNIQYTP SRDRQWHLYT VVDKPVSIQR |
| 1321 | MFLRTLVRQP TTNEVFTACQ GLGMEAPQAQ WTMSFTSRSI LRSLVAAMEE LELNMHNATV |
| 1381 | KSDHAHMYLC ILREQQIDDL VPYPKRVDIE AGQEEVAIGR ILEELAREIH ASVGVKMHRL |
| 1441 | NVCEWEVKLW MTSCGQANGA WRVVITNVTG HTCAVHTYRE LEDASKHGVV YHSVSVQGPL |
| 1501 | HGVLVNAVYQ SLGVLDRKRL LARRSNTTYC YDFPLAFETA LEQIWASQFT GTGKLKCNVL |
| 1561 | VKATELVFSD QKGSWGTPLV PVDRPAGLND IGMIAWTMEL STPEFPSGRT ILIVANDVTF |
| 1621 | KAGSFGPRED AFFYAVTDLA CTKKLPLIYL AANSGARIGV AEEVKSCFKV GWSDETSPEG |
| 1681 | GFQYVYLSPE DYTHIASSVI AHELKLSNGE TRWVIDAIVG KEDGLGVENL SGSGAIASAY |
| 1741 | SRAYKETFTL TYVTGRTVGI GAYLARLGMR CMQRVDQPII LTGFSALNKL LGREVYSSHI |
| 1801 | QLGGPKVMAT NGVVHLTVSD DLEGVSAILN WLSCIPPCIG GTLPILGPSD PTERPVEYFP |
| 1861 | ENSCDPRAAI SGSLDGNGKW LGGIFDKNSF VETLEGWART VVTGRAKLGG IPVGVIAVET |
| 1921 | QTVMQVIPAD PGQLDSHERV VPQAGQVWFP DSATKTAQAI LDFNREELPL FILAYWRGFS |
| 1981 | GGQRDLFEGI LQAGSTIVEN LRTYNQPVFV YIPMMGELRG GAWVVVDSQI NSDHIEMYAD |
| 2041 | RTAKGNVLEP EGIIEIKFRT KELLESMGRL DKQLITLKAK LQEARNSWNF GMVEDLQQQI |
| 2101 | KSREKQLLPI YTQIATRFAE LHDSSLRMAA KGVIREIVDW DKSRAYFYKR LRRRIAEGSL |
| 2161 | IKTVKDAAGD QLSHKSAMDL IKNWFLDSDI ARGKEDAWGN DEAFFAWKDD QGKYEEKLQE |
| 2221 | LRVQKVLVQL TNIGDSMSDL KALPQGLAAL LRKVEPSSRG QIIEELRKVI S |

Computational domain analysis of J. curcas ACCase protein sequence showed the presence of the following signatures:

TABLE 3
Computational domain analysis of J. curcas ACCase protein sequence

| | |
|---|---|
| Domain | Sequence |

| | |
|---|---|
| 1) Biotin | PIHSILIANNGMAAVKFMRSIRTWAYETfgnekaillVAMATPEDMkiNAEHIRIADQFV |
| carboxylation | EVPGGTNNNNYANVQLILEMAEGTRVDAVWPGWGHASENPELPDALSAKGIVFLGPPATS |
| domain | MAALGdkigssliaqaadvptlpwsgshvkippescliaipdevyreacvytteeaiasc |
| 50-557aa | qvvgypamikaswggggkgirkvhnddevralfkqvqgevpgspifimkvasqsrhlevq |
| | llcdqhgnvaalhsrdcsvqrrhqkiieegpitvapletvkkleqaarrlaksvnyvgaa |
| | tveylysmetgeyyflelnprlqvehpvtewiaevnlpaaqvavgmgiplwqipeirrfy |
| | gvengggydawrktsvvatpfdfdkaestrpKGHCVAVRVTSEDPDDgFKPTSGKVQELS |
| | FKSKPNVWAYFSVKSGGGIHEFSDSQFGHVFAFGESRALAIANMVLGLKEIQIRGeIRTN |
| | VDYSIDLLHASDYRDNKIHTGWLDSRIA |
| 2). ATP-grasp fold | ESCLIAIPDEVYREACVYTTEEAIASCQVVGYPAMIKASWGGGGKGIRKVHNDDEVRALF |
| profile 203-397aa | KQVQGEVPGSPIFIMKVASQSRHLEVQLLCDQHGNVAALHsrdcSVQRR------HQKII |
| | EEGPITVAPLETVKKLEQAARRLAKSVNYVGAATVEYLYSMETgEYYFLELNPRLQVEHP |
| | VTEwIAEVNLPAAQVAVGMGI |
| 3)BIOTINYL_LIPOYL | LLQNDHDPSKLVAETPckLLRFLVLDGSHIEADTPYAEVEVMKMCMPLLSPASGVLQFK |
| signature 639-757a | MSEGQAMQAGELIARL |
| 4) Acetyl-coenzyme A | FFYAVTDLACTKKLPLIYLAANSGARIGVAEEVKSCFKVGWSDETSPEGGFQYVYLSPED |
| carboxyltransferase | Y-----------------------------------THIASSVIAHELKLSNGETRWVID |
| domain N-terminal | DAIVGKEDGLGVENLSGSGAIASAYSRAYKETFTLTYVTGRTVGIGAYLARLGMRCMQRV |
| region 1631-1811aa | VDQPIILTGFSALNKLLGREVYSSHIQLGGPKVMATN |
| 5) Acetyl-coenzyme | 758-1501aa |
| A central domain | |
| 6) Acetyl-coenzyme | GVVHLTVSDDLEGVSAILNWLSCIPPCIGGTLPILGPSDPTERPVEYFPE------NSCD |
| A carboxyl- | PRAaisgsldgngkWLGGIFDKNSFVETLEGWARTVVTGRAKLGGIPVGVIAVETQTVMQ |
| transferase | QVIPADPGQldshervvpqagqvwfpdsatKTAQAILDFNREELPLFILAYWRGFSGGQR |
| domain C-terminal | RDLFEGILQAGSTIVENLRTYNQPVFVYIpmMGELRGGAWVVVDSQINSDHIeMYADRTA |
| region profile | KGNVLEPEGIIEIKFRTKEL------LESMGRLDKQLITLKAKLQEARNSwnfgmvedLQ |
| 1812-2126aa | QQIKSREKQLLPIYTQIATRFAELHDSS |
| 7) | 1598-2155aa |
| Carboxyl_transferase | |
| domain | |

Further support for the functional localization of the predicted protein is found in the observations that: 1) The cloned sequences encoding the above mentioned predicted protein, lack any membrane spanning regions, suggestive of the protein to be cytosolic, and not membrane localized or secreted. J. curcas ACCase is a purely soluble protein without any membrane spanning regions. 2) Absence of the chloroplast transit peptide suggests the protein is not targeted to the chloroplast. The inferred results implicate the cloned sequences encode the homomeric ACCase sequences.

The following examples illustrate embodiments of the invention. It will be appreciated by one of skill in the art that the techniques disclosed in the examples which follow represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Isolation and Identification of ACCase Gene in Jatropha

The mature seeds of Jatropha curcas were obtained from Andhra Pradesh, South India. The seeds were germinated in natural fields. Very young leaves were collected from 2-3 month old seedlings. The material was stored at −70° C. until use. The genomic DNA from above leaf material was extracted following methods provided with Sigma Gen Elute™ Plant Genomic DNA miniprep kit (Sigma, USA).

### Example 2

### Formation of cDNA Clones Encoding ACCase

Design of degenerate oligonucleotide primers: The amplification of cytosolic ACCase gene from Jatropha curcas L. using degenerate primer strategy by PCR and its partial sequence is presented herein. Multiple primers with low degeneracy rate, particularly at the 3′ end, and in the intermediate fragments were designed based on the conserved sequence motifs of ACCase gene family of Zea mays (gi|1045304), Oryza sativa (japonica cultivar-group) (gi|3753346), Medicago sativa (gi|495724), Arabidopsis thaliana (gi|501151), Brassica napus (gi|12057068), Triticum aestivum (gi:514305) and Glycine max (clones 513 and 1221) (gi|1066856). The sequences were aligned using Clustal W. Twenty three combinations of degenerate oligonucleotide primers both in reverse and forward directions were designed and tested on the genomic DNA of J. curcas.

Protein sequences related to ACCase from higher plants were retrieved from the non-redundant public sequence NCBI databases using Arabidopsis thaliana, Glycine max, Medicago sativa, Trticum vulgare, sequences as a query in BLASTp searches. Multiple sequence alignment of ACCase sequences done using CLUSTALW. Design of degenerate oligonucleotide primers was based on the information derived from multiple sequence alignment and conserved domain annotation of the cytosolic ACCase gene family. Conserved blocks of peptide sequences were selected as sites for amplification, using the data obtained from multiple sequence alignments. Following this the peptide were backtranslated to generate degenerate nucleotide sequence corresponding to the peptide, using standard codon frequency tables Annealing temperature (Tm) and propensity to form self complementary hair-pins and primers was analyzed using FastPCR.

TABLE 4

List of degenerate primers designed using
multiple sequence alignment that were used
in amplification of cytosolic ACCase from
Jatropha curcas L.

| | | |
|---|---|---|
| | Se- | Prime |
| Primer sequence 5′-3′ | quence | type |

| | | | |
|---|---|---|---|
| ME 58 | AATGGAATGGCRGCWGTSAAGTTTATACG | For- | Degen- |
| | | ward | erate |
| ME 70 | CATGTTTTTGCDTTTGGRGAATC | For- | Degen- |
| | | ward | erate |
| ME 24 | GGCATGCACATCTTCATRACCTC | Re- | Degen- |
| | | verse | erate |
| ME 23 | GAGGTSTAYAGCTTYCASATGC | For- | Degen- |
| | | ward | erate |
| ME 25 | CTGAAGDATTCCTTCRAAVAG | Re- | Degen- |
| | | verse | erate |
| ME 86 | CTCATYTGRTTGTTGATSCC | Re- | Degen- |
| | | verse | erate |
| ME 59 | AGCAAGWCCTTGWGGYAGAGCTTG | Re- | Degen- |
| | | verse | erate |
| ME 71 | GAGGTBTAYAGCTCTCAGATGCAACT | For- | Degen- |
| | | ward | erate |

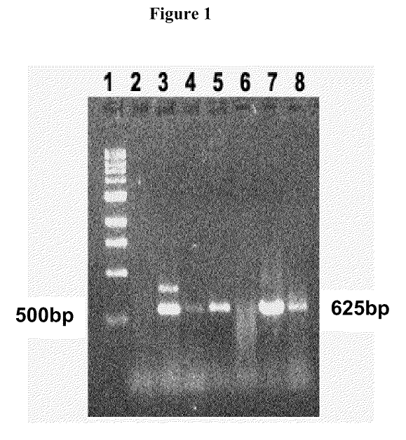

Amplification of 625 bp conserved domain: In order to characterise the Jatropha ACCase gene, amplification of the part of the gene with PCR is done using degenerate nucleotide primers. Using degenerate primers ME23 (5′-GAGGTSTAYAGCTTYCASATGC-3′ forward primer) and ME25 (5′-CTGAAGDATTCCTTCRAAVAG-3′ reverse primer), approximately 625 bp intermediate fragment was amplified from genomic DNA of Medicago sativa, Arabidopsis and Jatropha curcas (FIG. 1) indicating that it was indeed a conserved motif. The amplification was performed in a Eppendorf Gradient Thermal Cycler (96 wells) system for 35 cycles with 45 s at 94° C., 45 s at 55° C. and 1 min at 72° C. The first ten cycles used a Touch Down program of 65° C.-55° C. by decreasing 1° C. every cycle and for the remaining 25 cycles, 55° C. annealing temperature was maintained. After the final cycle the amplification was extended for 7 min at 72° C. Products of degenerate primer PCR reactions were subjected to gel electrophoresis (1% agarose, with TAE as the running buffer) according to Sambrook et al (1989) and DNA fragments of 625 bp in the length were recovered from the gel using QIA Quick gel extraction kit (Qiagen, Valencia, Calif.). This product was then cloned into pGEM-T Easy Vector System I (Promega Corp, USA) and transformed into Escherichia coli DH5α (Gibco BRL). The construct was designated pGEM:J1 clone.

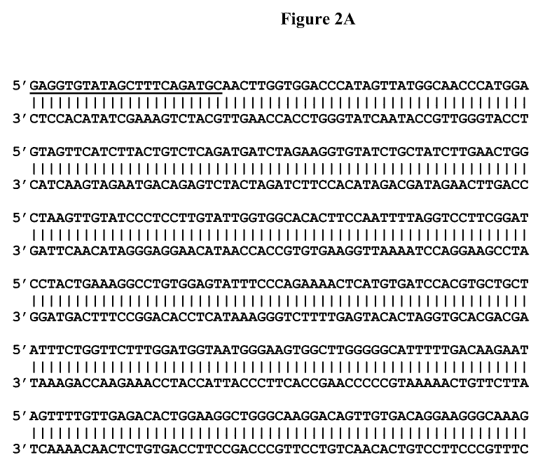

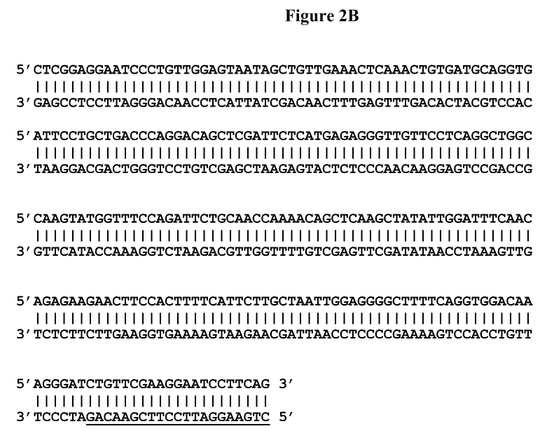

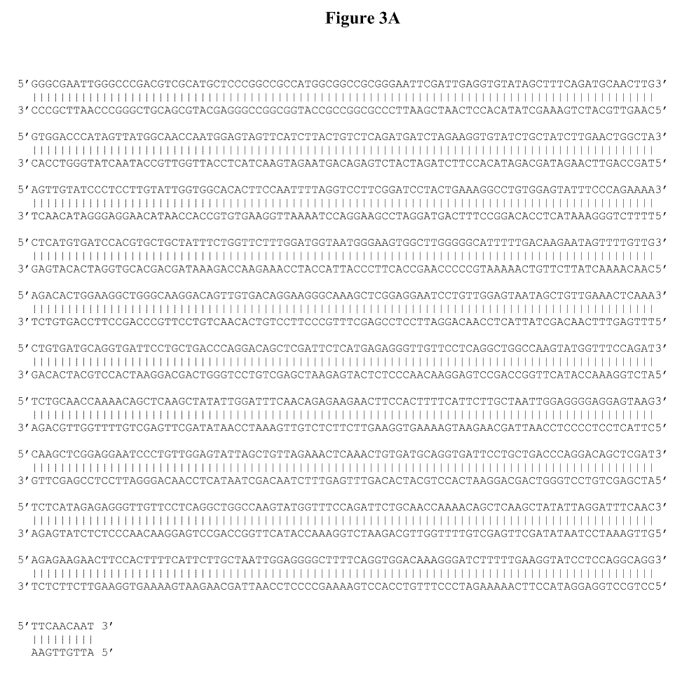

Sequencing of pGEM: J1 clone: Positive recombinant colonies were isolated and plasmid DNA prepared. Sequencing was carried out using M13 forward and reverse universal primers using ABI Prism Automated sequencing. For 597 bp sequencing results see FIG. 2.

Sequence similarity and comparison among various ACCase gene family members: The coding nucleotide sequence of clone pGEM:J1 containing 625 bp conserved motif of ACCase gene from Jatropha curcas was subjected to BLASTn (Basic Local Alignment Search Tool) against GenBank sequences.

A database search with BLASTn (National Center for Biotechnology Information databases) showed relatively high similarity with other ACCase gene family. The percentage similarity with Glycine max (gi|992916) was 84%; Medicago sativa (gi|495724) 84%; Phaseolus vulgaris (gi|7839251) 84%; and Elaeis guineensis micro satellite (gi|12053787) 100%.

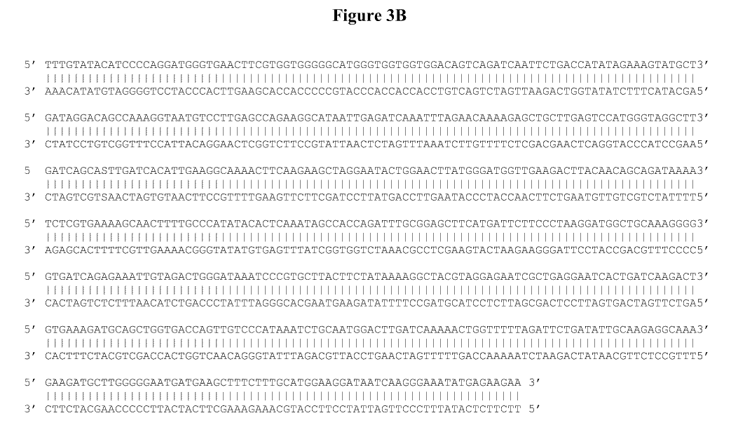

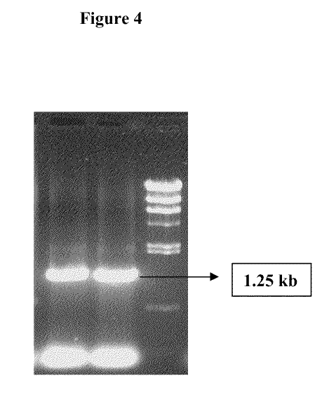

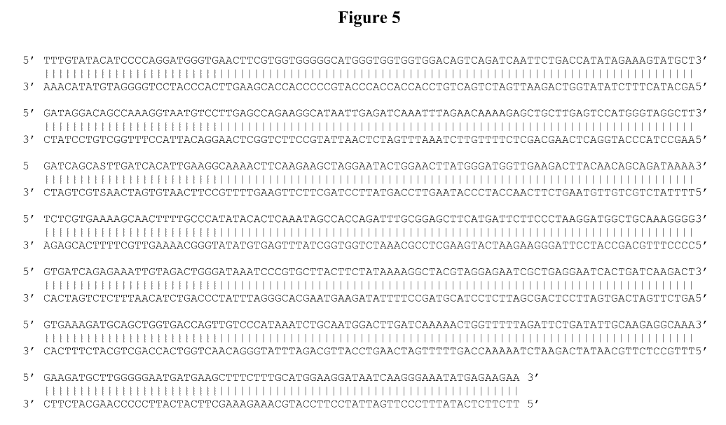

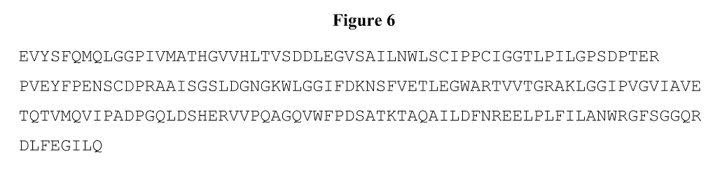

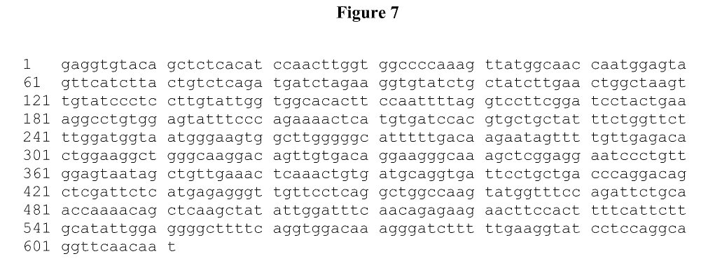

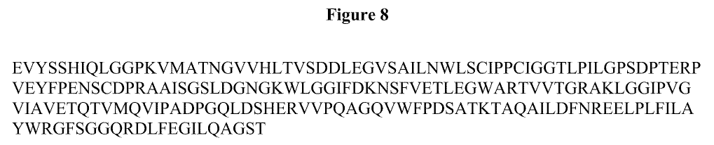

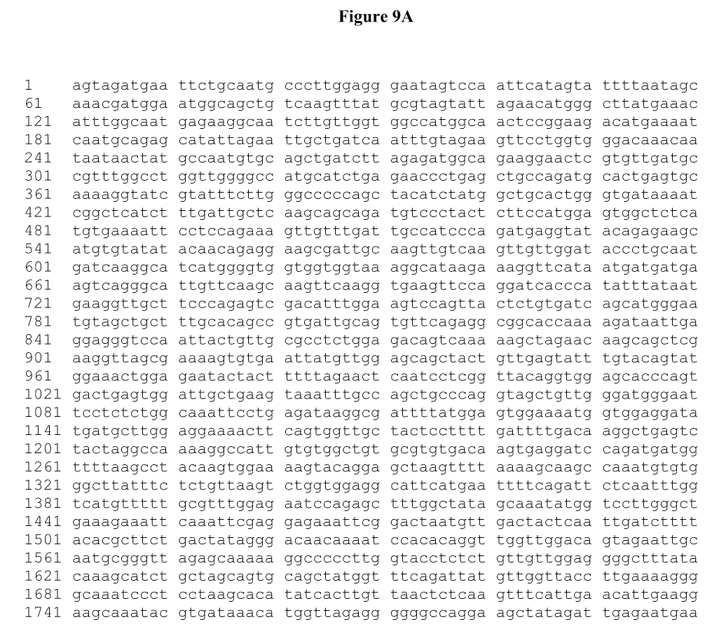

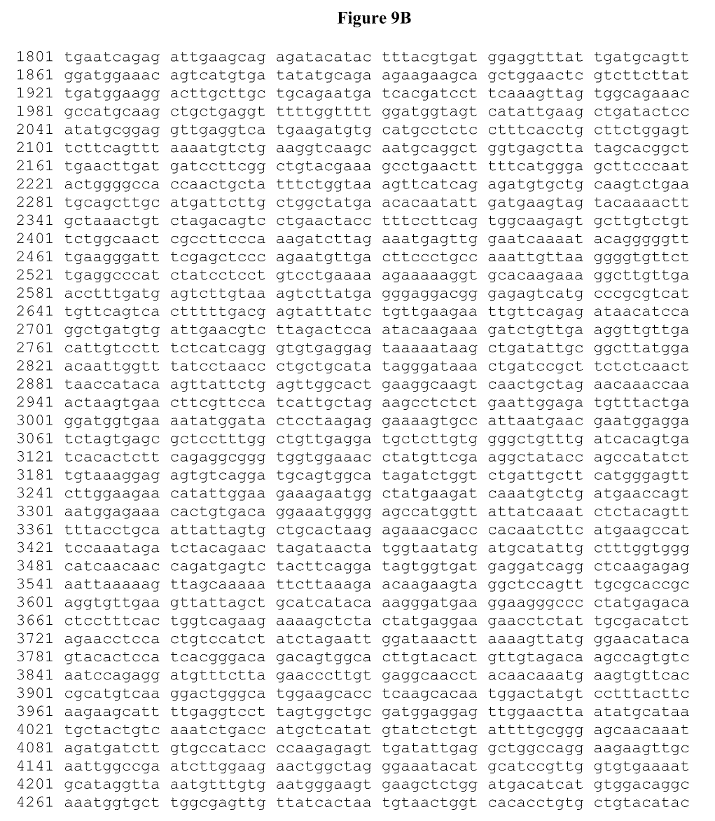

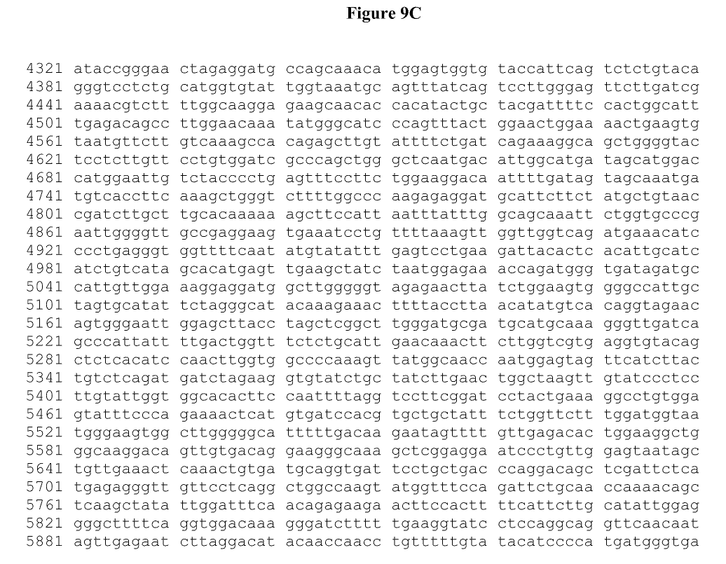

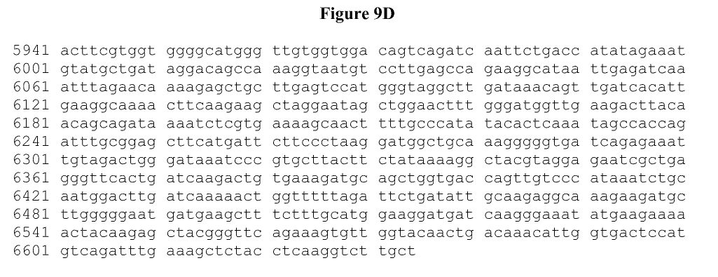

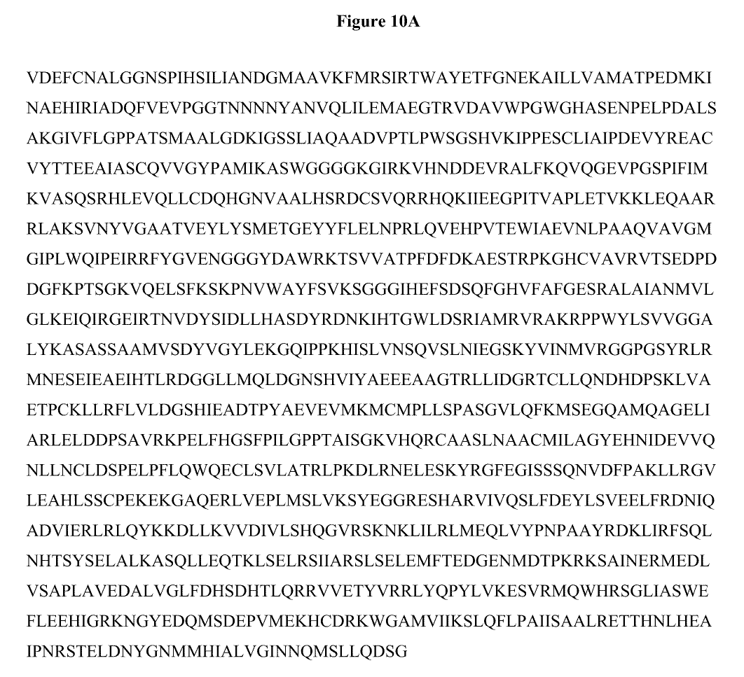

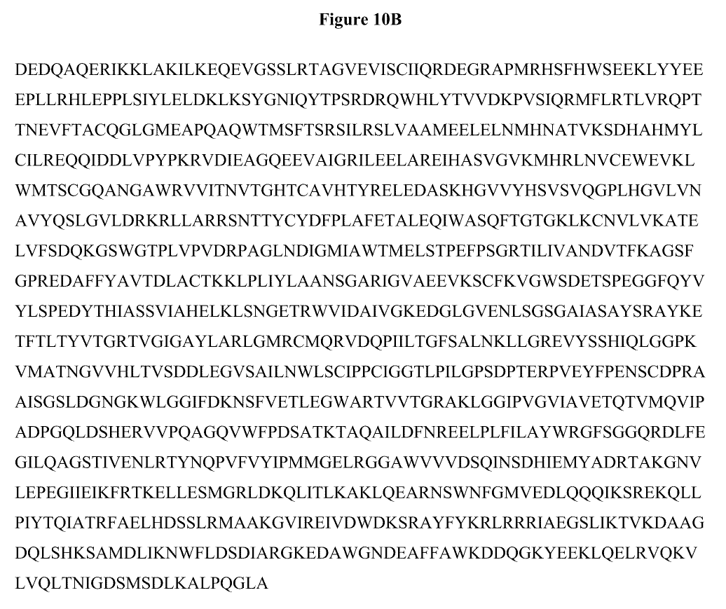

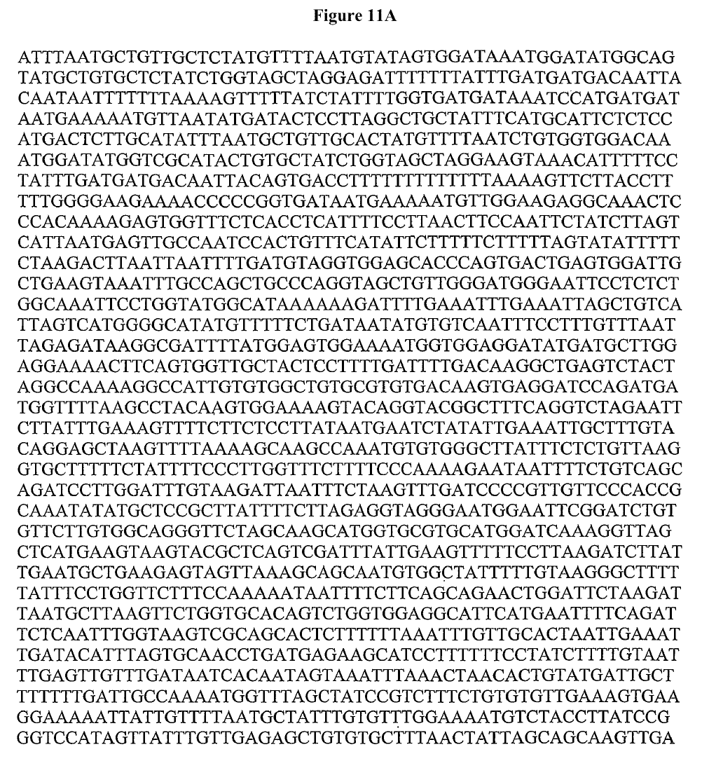

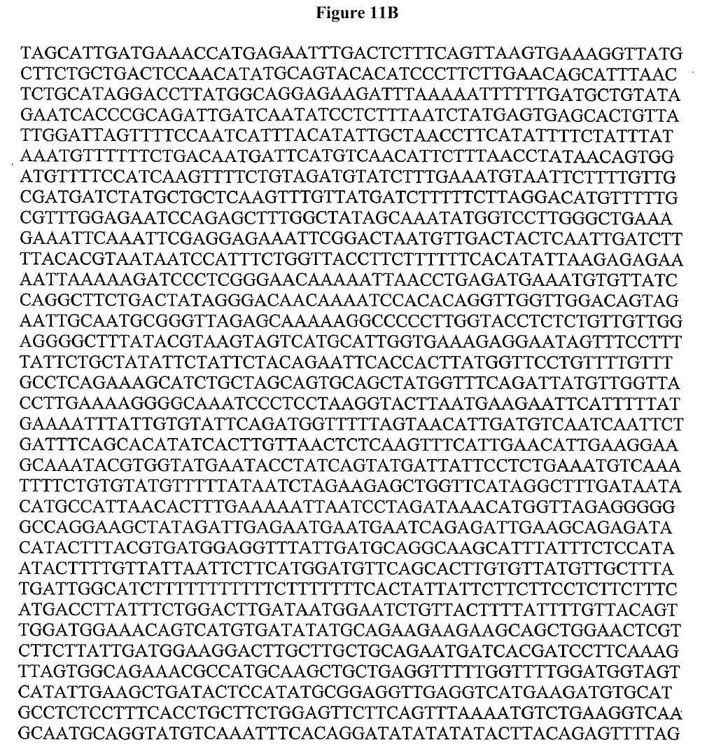

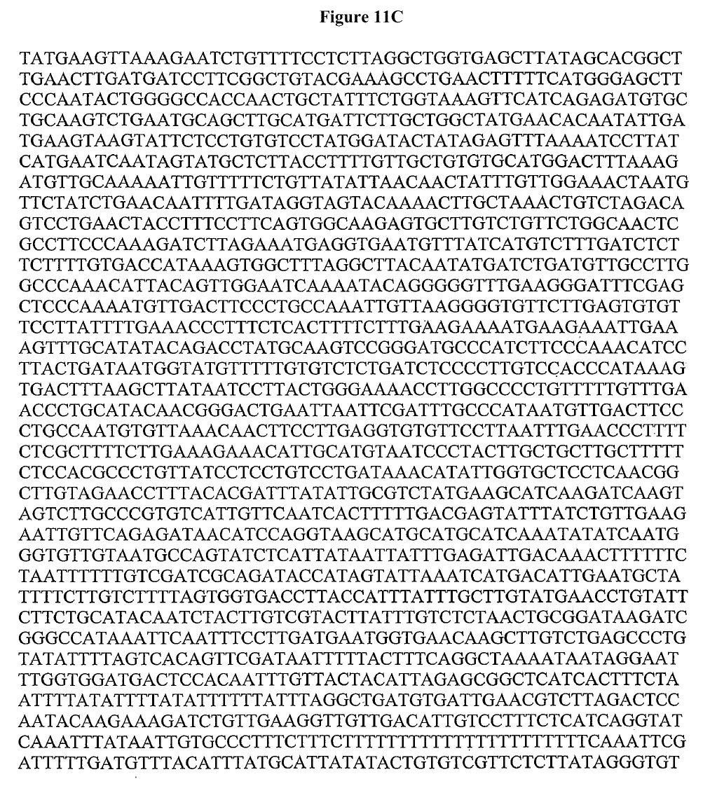

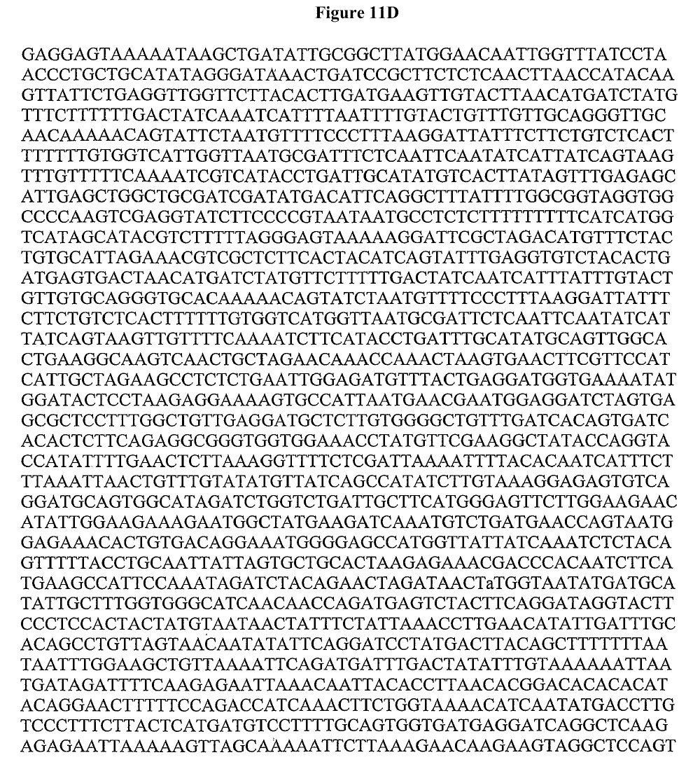

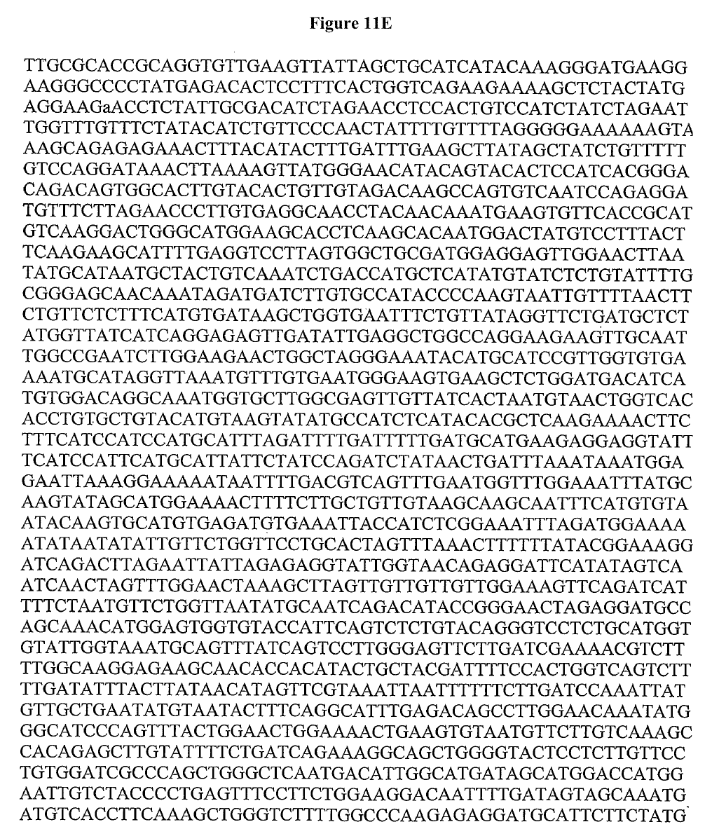

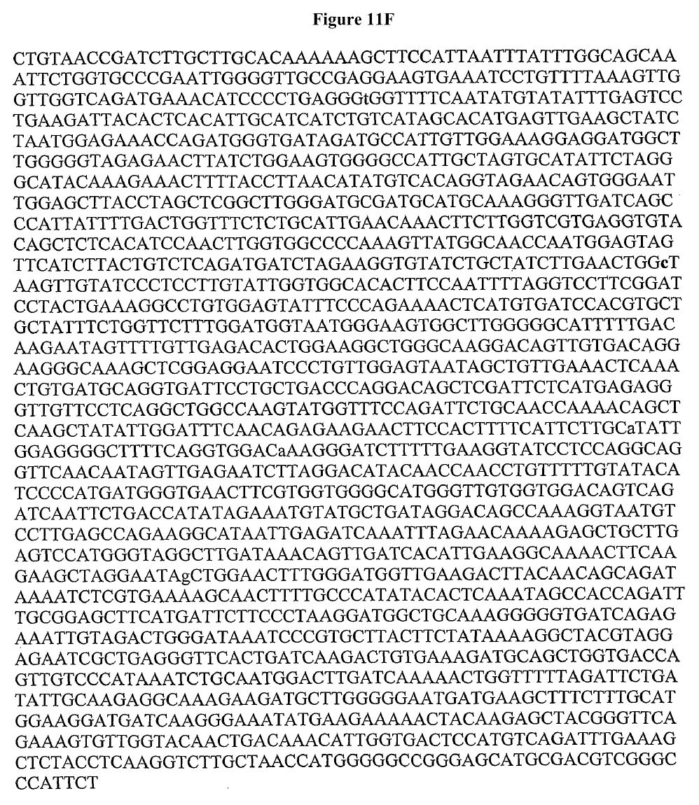

Amplification of the 1.25 kb intermediate fragment from ACCase gene: With the sequence of 597 bp intermediate fragment from conserved sequence motif of J. curcas (FIG. 2), gene specific forward primers at 597 bp region and degenerate nucleotide reverse primer from 3′ end were designed. Gene specific forward primer (ME-50: 5′ GTCTCAGATGATCTAGAAGGTGTATC 3′) and degenerate reverse primer (ME-59: 5′AGCAAGWCCTTGWGGYAGAGCTTG3′) were designed based on conserved domains of Medicago sativa, Arabidopsis and Glycine max at the 3′ region. The product of ME50 and ME 59 was a 1.3 kb fragment that was amplified and gel eluted as described earlier. The PCR product was sequenced and designated J-2. (FIG. 3). Based on sequence results of above 1.3 kb fragment gene specific forward (ME-75: 5′ GTGGACCCATAGTTATGGCAACC 3′) and reverse primer (ME-76: 5′ AGAAAGCTTCATCATTCCCCCAAG 3′) were designed to amplify a 1.25 kb fragment of ACCase gene towards 3′ end. Results are shown in FIG. 4. The amplification was performed in a BioRad i-Cylcer Thermal Cycler (96 wells) system for 33 cycles with 45 s at 95° C., 45 s at 54° C. and 6 min at 72° C. The first eight cycles used a Touch Down program of 62° C.-54° C. by decreasing 1° C. every cycle and for the remaining 25 cycles 54° C. annealing temperature was maintained. After the final cycle the amplification was extended for 20 min at 72° C. Products of PCR reactions were subjected to gel electrophoresis (1% agarose, with TAE as the running buffer) according to Sambrook et al (1989) and DNA fragments of 1.25 kb in the length were recovered from the gel using QIA Quick gel extraction kit (Qiagen). The PCR product confirmed the presence of a 1.25 kb fragment (FIG. 4).

### Example 3

### Genomic DNA Preparation

Jatropha curcas genomic DNA was prepared from leaf (Grown in DALSC) and callus using Genelute plant genomic kit (Sigma). Initially degenerate primers ME 23×ME 25 (towards 3′ end) and ME70×ME24 (towards 5′ end) primers were used to amplify the product from genomic DNA by Touch Down polymerase chain reaction (Table 5). After running PCR product on gel right bands were eluted (Qiagen kit) and cloned in pGEMT Vector (Promega) and sequenced. The results were analysed using BLAST (NCBI). Further degenerate primers ME58×ME86, ME 71×ME 59 were used to get rest of the sequence. Then, gene specific primers were designed from the sequence data, and other unidentified areas were explored. The following amplification programs were used for amplifying cytosolic ACCase from J. curcas.

TABLE 5
Touch-down PCR program used in amplifying cytosolic ACCase from J. curcas L.

| Program 1: | Program 2: | Program 3: |
|---|---|---|
| 94° C., 3 min, | 94° C., 3 min, | 94° C., 3 min, |
| 94° C., 45 sec, | 94° C., 45 sec, | 94° C., 45 sec, |
| 62° C., 1 min (Touch down, | 58° C., 1 min (Touch down, | 58° C., 1 min (Touch down, |
| reduce 1° C. every cycle) | reduce 1° C. every cycle) | reduce 1° C. every cycle) |
| 72° C., 3 min | 72° C., 3 min | 72° C., 3 min |
| Repeat 8 cycles' | Repeat 8 cycles' | Repeat 8 cycles' |
| 94° C., 45 sec, | 94° C., 45 sec, | 94° C., 45 sec, |
| 55° C., 1 min | 53° C., 1 min | 61° C., 1 min |
| 72° C., 3 min | 72° C., 3 min | 72° C., 3 min |
| Repeat 22 cycles (step 6-8) | Repeat 22 cycles (step 6-8) | Repeat 22 cycles (step 6-8) |
| 72° C., 20 min | 72° C., 20 min | 72° C., 20 min |

### Example 4

### RNA and cDNA Preparation (Reverse Transcription)

Total RNA was isolated from leaf/callus by Trizol/Trireagent/Plant RNA extract reagent and Reverse transcription was done using gene specific reverse primers by MuMLV (Fermentas). PCR was done using gene specific primers (See Table 6) and products were cloned

TABLE 6

List of gene specific primers used in
amplification of cytosolic ACCase gene.

| Primer | |
|---|---|
| No | Primer sequence 5′-3′ |
| ME-91 | ACTCCTCAAGGCTATAAGAACCTTACA |
| ME-92 | CTTGGGGTATGGCACAAGATCATCT |
| ME-93 | TGTGACAAGTGAGGATCCAGATGA |
| ME-94 | TGGATGTTATCTCTGAACAATTCTTCA |
| ME-95 | TGGCAATGAGAAGGCAATCTTGT |
| ME-96 | TGAAAATTCATGAATGCCTCCACCAGA |
| ME-97 | GGAACATACAGTACACTCCATCACG |
| ME-98 | AGCAAGATCGGTTACAGCATAGAAGA |
| ME-99 | AGCTGGGCTCAATGACATTGGCA |

### Example 5

### Exploration of Stop Codon and 3′UTR

Total RNA was isolated from leaf as above and Reverse transcription was done using AMBION kit as per the manufacturer's protocol. Gene specific primers for 3″ RACE are listed below (Table 7). Amplification product cloned in pGEM T/A vector and sequenced.

TABLE 7

List of gene specific primers for cloning stop
codon and 3′UTR using 3′ RACE technology.

| | |
|---|---|
| Primer No | Primer sequence 5′-3′ |

| | |
|---|---|
| ME 119 | GATGCTTGGGGGAATGATGA |
| ME 120 | ACAGCCATGGATGGCATAGG |

Exploration of 5′UTR and start codon by Thermal Asymmetric Interlaced PCR (TAIL PCR) were carried out according to: (1). Liu Y G, Mitsukawa N, Oosumi T, Whittier R F: Efficient isolation and mapping of Arabidopsis thaliana T-DNA insert junctions by thermal asymmetric interlaced PCR. Plant J 1995, 8:457-463; and (2) Hanhineva K J* and Kärenlampi, S.O. Production of transgenic strawberries by temporary immersion bioreactor system and verification by TAIL-PCR, BMC Biotechnology 2007, 7:11)

### Example 6

### Vector Construction

Step 1: Construction of pCAME from pCA

TABLE 8

List of primers including TAIL PCR used
in amplifying 5′ UTR

| | | |
|---|---|---|
| Primer No | Primer sequence 5′-3′ | Comment |

| | | |
|---|---|---|
| ME-193 | 5′GTAGGGACATCTGCTGCTTGA 3″ | Designed by inventors |
| | | based on original sequence |
| ME-194 | 5′GCTTGAGCAATCAAAGATGAGCCG 3″ | Designed by inventors |
| | | based on original sequence |
| ME-195 | 5′AGCCGATTTTATCACCCAGTG 3′ | Designed by inventors |
| | | based on original sequence |
| ME-185 | 5′ NTCGASTWTSGWGTT 3′ | Liu et al., 1995 Hanhineva |
| | | and Kärenlampi, 2007 |
| ME-186 | 5′ NGTCGASWGANAWGAA 3′ | Liu et al., 1995 Hanhineva |
| | | and Kärenlampi, 2007 |
| ME-187 | 5′ WGTGNAGWANCANAGA 3′ | Liu et al., 1995 Hanhineva |
| | | and Kärenlampi, 2007 |
| ME-188 | 5′ WGCNAGTNAGWANAAG 3′ | Liu et al., 1995 Hanhineva |
| | | and Kärenlampi, 2007 |
| ME-189 | 5′ AWGCANGNCWGANATA 3′ | Liu et al., 1995 Hanhineva |
| | | and Kärenlampi, 2007 |

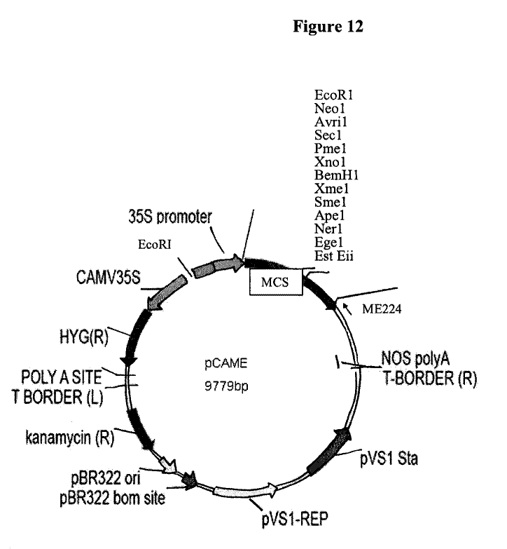

MBIA 1303: Binary vectors were purchased from pCAMBIA. (www.cambia.org/daisy/cambia/585.html#dsy585_promoter_cloner). pCAMBIA 1303 vectors are kanamycin (prokaryotic selection) and Hygromycin (eukaryotic selection) driven by CaMV/35s. pCAMBIA 1303 modified by removing gus A and GFP and added unique multiple doing site and cloned additional CaMV35S promoter in opposite direction to drive the gene of interest. See FIG. 12.

Step 2: Construction of pCA-ME with Jatropha ribulose 1,5-bisphosphate carboxylase small subunit (transit peptide or Signal sequence): This short signal sequence present in chloroplast are capable of translocating a protein operably linked to the transit peptide, to a plant cell plastid. In plants, fatty acid biosynthesis occurs prdominantly in storma of plastids. The first step of fatty acid synthesis is conversion of Acetyl co A into Mlaonyl CoA through Acetyl Co A carboxylase. To target the cytosolic ACCase in chloroplast, transit peptide need to be tagged with cytosolic ACCase.

Genomic DNA of Jatropha curcas was used to amplify transit peptide with restriction sites.

Primers: ME-220 is a forward primer for Jatropha curcas transit peptide with Hind III having the sequence 5′-CGAAGCTTATGGCTTCCTCAGTTCTTTC-3′ and ME-221 is a reverse primer for Jatropha curcas transit peptide with Avr II 5′-ATCCTAGGCATGCATTGCACTCTTCC-3′.

Jatropha ribulose 1,5-bisphosphate carboxylase small subunit 174 bp can be accessed at GenBank Accession No: EU395776. The sequence is in Table 9.

TABLE 9
Jatropha ribulose 1,5-bisphosphate carboxylase small subunit 174 b

| | |
|---|---|
| 1 | atggcttcct cagttctttc ctctgcagca gttgccaccc gcagcaatgt tgctcaagct |
| 61 | aacatggttg cacctttcac tggccttaag tcagctgcct cattccctgt ttcaaggaag |
| 121 | caaaaccttg acatcacttc cattgccagc aacggcggaa gagtgcaatg catg |

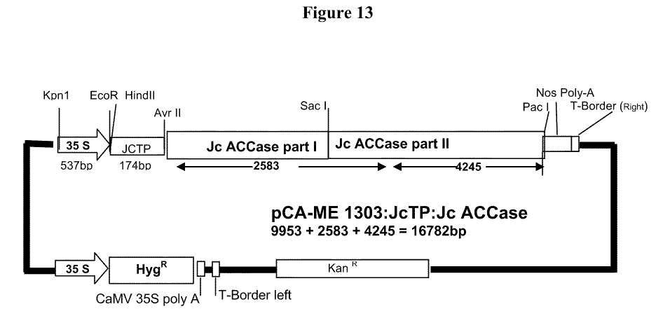

Step 3: Cloning of J. curcas ACCase cDNA in two parts: RNA was isolated from young leaf/early fruit by hot borate method as mentioned in Wu et al. 2003. Reverse Transcription was done using gene specific reverse primers Superscript Reverse transcriptase First Strand cDNA synthesis (Invitrogen) kit by following manufacturer's protocol. The following primers were used: ME-216: Forward primer 1 for Jatropha ACCase 5′ part I with restriction site Avr II: 5′ ATACCTAGGATGCAGCATGTTGGAAGCACAAAG 3′; ME-217: Reverse primer1 for Jatropha ACCase 5′ part I with restriction site SacI: 5′ AACATTCTGGGAGCTCGAAATCCCTTC 3′; ME-218: Forward primer 2 for Jatropha ACCase part II with restriction site Sac I: 5′ GAAGGGATTTCGAGCTCCCAGAATGTT 3′; and ME-219: Reverse primer for Jatropha ACCase part II with restriction site Pac I: 5′-CCGTTAATTAATCAACTGATGACCTTTCGAAGCTCTTC-3′. The construct is shown in FIG. 13.

All compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention. Indeed, various modifications of the described modes for carrying out the invention which are understood by those skilled in the art are intended to be within the scope of the claims.

All publications and patent applications cited in this specification are herein incorporated by reference as if each individual publication or patent application is specifically and individually indicated to be incorporated by reference in its entirety.

1. An isolated and purified polypeptide comprising an active acetyl-CoA carboxylase (ACCase) from Jatropha curcas L.

2. The polypeptide of claim 1, wherein said polypeptide is selected from the group consisting of SEQ ID NO: 2, SEQ ID NO:9, SEQ ID NO:11, SEQ ID NO:13 and fragments thereof.

3. The polypeptide of claim 0, wherein said polypeptide comprises SEQ ID NO: 2, as listed in Table 2.

4. The isolated and purified polypeptide of claim 0, wherein the polypeptide is encoded by a nucleic acid sequence comprising SEQ ID NO:1, as listed in Table 1.

5. An isolated and purified nucleic acid coding for an acetyl-CoA carboxylase (ACCase) gene or fragment thereof from Jatropha curcas L and having a sequence having at least 85% similarity to a sequence of Jatropha curcas acetyl-CoA carboxylase (ACCase).

6. The nucleic acid of claim 5, wherein said nucleic acid is selected from the group consisting of SEQ ID NO: 1, SEQ ID NO:4, SEQ ID NO:10, SEQ ID NO:12 and fragments thereof.

7. A plant containing a recombinant nucleic acid construct comprising a nucleic acid encoding a nucleic acid encoding a cytosolic Jatropha curcas ACCase operably linked to a promoter, wherein said plant produces seeds that exhibit a statistically significant increase in oil content as compared to seeds produced by a corresponding plant lacking said nucleic acid construct.

8. A method of producing a genetically modified Jatropha curcas plant, comprising:
a) providing a Jatropha curcas plant;
b) introducing an expression cassette comprising a nucleic acid coding for ACCase and optionally, a transit peptide, into the plant;
c) detecting increased activity or increased expression of the ACCase in said plant in order to determine if the plant has been genetically modified; and
d) regenerating said genetically modified Jatropha curcas plant.

9. The method of claim 8, wherein said ACCase is a cytosolic Jatropha curcas ACCase.

10. The method of claim 8, wherein said ACCase is operably linked to a promoter capable of expression in Jatropha curcas.

11. The method of claim 0, wherein said ACCase is expressed in the genetically modified Jatropha curcas plant.

12. The method of claim 0, wherein said promoter is an inducible promoter.

13. The method of claim 0, wherein said promoter is selected from the group consisting of a CaMV 35S promoter, a nopaline synthase (NOS) promoter, and an endosperm-specific promoter.

14. The method of claim 0, wherein said endosperm-specific promoter is selected from the group consisting of a beta-phaseolin promoter, a napin promoter and an ubiquitin promoter.

15. The method of claim 8, wherein said transit peptide is a chloroplast transit peptide positioned between the promoter and the ACCase.

16. The method of claim 8, wherein the expression cassette is introduced into the plant by a method selected from the group consisting of: protoplast transformation, Agrobacterium mediated transformation, electroporation, and microprojectile bombardment.

17. The method of claim 8, wherein the expression cassette further comprises one or more of plant transcriptional termination and polyadenylation signals and translational signals linked to the 3′ terminus of a plant ACCase gene.

18. The method of claim 8, wherein said genetically modified Jatropha curcas plant produces seed with increased oil content.

19. The method of claim 0, wherein said oil content is increased by 1.2-20 fold compared to seed produced by a naturally occurring Jatropha curcas plant.

20. A seed from a genetically modified Jatropha curcas plant produced by the method according to claim 8, wherein said genetically modified Jatropha curcas plant produces seed with increased oil content compared to seed produced from a naturally occurring Jatropha curcas plant.


SEQUENCE LISTING (58 sequences)

SEQ ID NO: 1 - DNA, 7715 bp, J. curcas
  misc_feature (535)..(535): n is a, c, g, or t
gagtttggtgtttgaagtatcgggatagtattgtttttagcagcatagtgatgggaagtttctatgctgttctttttatgggtggtttttaaatttttattgatcaaattttagttatggatttgggaatctaattgtgctcttttgtgggggcgaatgactattttatgttattgggcactttttcatcgttatcatcaatatcctaggattctgcacctgagatactttgacttttaaactgtgtttggtttcttataacttaagtggaatatactaatgaattggggttcggttttagtgatttaactctgattatcaaacaactacacgatcaagtctagagtttatagtcccaggaagtaaagaagtctacatcaattccttatttgcaaataaagttgttaaattgttctgtcaacttttgctcacgaactatgaacaaatgatattactaaggaaggcctacccgaaaagtaaaactgtatttaagtataagaaaacgattgacgttgagaatgatttaatttgaagnctcaattttggtaacaaaagtcctatctatgcagcatgttggaagcacaaaggagaccaccggaaccggtgggtgttgctcgtgtaatggttacataaatggggtagtttcaatgagaagtcctgctacaatatctgaagtggatgaattctgccatgctcttggagggaatagtccaattcatagtattttaatagcaaacaatggaatggcagctgtcaagtttatgcgtagtattagaacatgggcttatgaaacatttggcaatgagaaggcaatcttgttggtggccatggcaactccggaagacatgaaaatcaatgcagagcatattagaattgctgatcaatttgtagaagttcctggtgggacaaacaataataactatgccaatgtgcagctgatcttagagatggcagaaggaactcgtgttgatgccgtttggcctggttggggccatgcatctgagaaccctgagctgccagatgcactgagtgcaaaaggtatcgtatttcttgggcccccagctacatctatggctgcactgggtgataaaatcggctcatctttgattgctcaagcagcagatgtccctactcttccatggagtggctctcatgtgaaaattcctccagaaagttgtttgattgccatcccagatgaggtatacagagaagcatgtgtatatacaacagaggaagcgattgcaagttgtcaagttgttggataccctgcaatgatcaaggcatcatggggtggtggtggtaaaggcataagaaaggttcataatgatgatgaagtcagggcattgttcaagcaagttcaaggtgaagttccaggatcacccatatttataatgaaggttgcttcccagagtcgacatttggaagtccagttactctgtgatcagcatgggaatgtagctgctttgcacagccgtgattgcagtgttcagaggcggcaccaaaagataattgaggagggtccaattactgttgcgcctctggagacagtcaaaaagctagaacaagcagctcgaaggttagcgaaaagtgtgaattatgttggagcagctactgttgagtatttgtacagtatggaaactggagaatactactttttagaactcaatcctcggttacaggtggagcacccagtgactgagtggattgctgaagtaaatttgccagctgcccaggtagctgttgggatgggaattcctctctggcaaattcctgagataaggcgattttatggagtggaaaatggtggaggatatgatgcttggaggaaaacttcagtggttgctactccttttgattttgacaaggctgagtctactaggccaaaaggccattgtgtggctgtgcgtgtgacaagtgaggatccagatgatggttttaagcctacaagtggaaaagtacaggagctaagttttaaaagcaagccaaatgtgtgggcttatttctctgttaagtctggtggaggcattcatgaattttcagattctcaatttggtcatgtttttgcgtttggagaatccagagctttggctatagcaaatatggtccttgggctgaaagaaattcaaattcgaggagaaattcggactaatgttgactactcaattgatcttttacacgcttctgactatagggacaacaaaatccacacaggttggttggacagtagaattgcaatgcgggttagagcaaaaaggcccccttggtacctctctgttgttggaggggctttatacaaagcatctgctagcagtgcagctatggtttcagattatgttggttaccttgaaaaggggcaaatccctcctaagcacatatcacttgttaactctcaagtttcattgaacattgaaggaagcaaatacgtgataaacatggttagaggggggccaggaagctatagattgagaatgaatgaatcagagattgaagcagagatacatactttacgtgatggaggtttattgatgcagttggatggaaacagtcatgtgatatatgcagaagaagaagcagctggaactcgtcttcttattgatggaaggacttgcttgctgcagaatgatcacgatccttcaaagttagtggcagaaacgccatgcaagctgctgaggtttttggttttggatggtagtcatattgaagctgatactccatatgcggaggttgaggtcatgaagatgtgcatgcctctcctttcacctgcttctggagttcttcagtttaaaatgtctgaaggtcaagcaatgcaggctggtgagcttatagcacggcttgaacttgatgatccttcggctgtacgaaagcctgaactttttcatgggagcttcccaatactggggccaccaactgctatttctggtaaagttcatcagagatgtgctgcaagtctgaatgcagcttgcatgattcttgctggctatgaacacaatattgatgaagtagtacaaaacttgctaaactgtctagacagtcctgaactacctttccttcagtggcaagagtgcttgtctgttctggcaactcgccttcccaaagatcttagaaatgagttggaatcaaaatacagggggtttgaagggatttcgagctcccagaatgttgacttccctgccaaattgttaaggggtgttcttgaggcccatctatcctcctgtcctgaaaaagaaaaaggtgcacaagaaaggcttgttgaacctttgatgagtcttgtaaagtcttatgagggaggacgggagagtcatgcccgcgtcattgttcagtcactttttgacgagtatttatctgttgaagaattgttcagagataacatccaggctgatgtgattgaacgtcttagactccaatacaagaaagatctgttgaaggttgttgacattgtcctttctcatcagggtgtgaggagtaaaaataagctgatattgcggcttatggaacaattggtttatcctaaccctgctgcatatagggataaactgatccgcttctctcaacttaaccatacaagttattctgagttggcactgaaggcaagtcaactgctagaacaaaccaaactaagtgaacttcgttccatcattgctagaagcctctctgaattggagatgtttactgaggatggtgaaaatatggatactcctaagaggaaaagtgccattaatgaacgaatggaggatctagtgagcgctcctttggctgttgaggatgctcttgtggggctgtttgatcacagtgatcacactcttcagaggcgggtggtggaaacctatgttcgaaggctataccagccatatcttgtaaaggagagtgtcaggatgcagtggcatagatctggtctgattgcttcatgggagttcttggaagaacatattggaagaaagaatggctatgaagatcaaatgtctgatgaaccagtaatggagaaacactgtgacaggaaatggggagccatggttattatcaaatctctacagtttttacctgcaattattagtgctgcactaagagaaacgacccacaatcttcatgaagccattccaaatagatctacagaactagataactatggtaatatgatgcatattgctttggtgggcatcaacaaccagatgagtctacttcaggatagtggtgatgaggatcaggctcaagagagaattaaaaagttagcaaaaattcttaaagaacaagaagtaggctccagtttgcgcaccgcaggtgttgaagttattagctgcatcatacaaagggatgaaggaagggcccctatgagacactcctttcactggtcagaagaaaagctctactatgaggaagaacctctattgcgacatctagaacctccactgtccatctatctagaattggataaacttaaaagttatgggaacatacagtacactccatcacgggacagacagtggcacttgtacactgttgtagacaagccagtgtcaatccagaggatgtttcttagaacccttgtgaggcaacctacaacaaatgaagtgttcaccgcatgtcaaggactgggcatggaagcacctcaagcacaatggactatgtcctttacttcaagaagcattttgaggtccttagtggctgcgatggaggagttggaacttaatatgcataatgctactgtcaaatctgaccatgctcatatgtatctctgtattttgcgggagcaacaaatagatgatcttgtgccataccccaagagagttgatattgaggctggccaggaagaagttgcaattggccgaatcttggaagaactggctagggaaatacatgcatccgttggtgtgaaaatgcataggttaaatgtttgtgaatgggaagtgaagctctggatgacatcatgtggacaggcaaatggtgcttggcgagttgttatcactaatgtaactggtcacacctgtgctgtacatacataccgggaactagaggatgccagcaaacatggagtggtgtaccattcagtctctgtacagggtcctctgcatggtgtattggtaaatgcagtttatcagtccttgggagttcttgatcgaaaacgtcttttggcaaggagaagcaacaccacatactgctacgattttccactggcatttgagacagccttggaacaaatatgggcatcccagtttactggaactggaaaactgaagtgtaatgttcttgtcaaagccacagagcttgtattttctgatcagaaaggcagctggggtactcctcttgttcctgtggatcgcccagctgggctcaatgacattggcatgatagcatggaccatggaattgtctacccctgagtttccttctggaaggacaattttgatagtagcaaatgatgtcaccttcaaagctgggtcttttggcccaagagaggatgcattcttctatgctgtaaccgatcttgcttgcacaaaaaagcttccattaatttatttggcagcaaattctggtgcccgaattggggttgccgaggaagtgaaatcctgttttaaagttggttggtcagatgaaacatcccctgagggtggttttcaatatgtatatttgagtcctgaagattacactcacattgcatcatctgtcatagcacatgagttgaagctatctaatggagaaaccagatgggtgatagatgccattgttggaaaggaggatggcttgggggtagagaacttatctggaagtggggccattgctagtgcatattctagggcatacaaagaaacttttaccttaacatatgtcacaggtagaacagtgggaattggagcttacctagctcggcttgggatgcgatgcatgcaaagggttgatcagcccattattttgactggtttctctgcattgaacaaacttcttggtcgtgaggtgtacagctctcacatccaacttggtggccccaaagttatggcaaccaatggagtagttcatcttactgtctcagatgatctagaaggtgtatctgctatcttgaactggctaagttgtatccctccttgtattggtggcacacttccaattttaggtccttcggatcctactgaaaggcctgtggagtatttcccagaaaactcatgtgatccacgtgctgctatttctggttctttggatggtaatgggaagtggcttgggggcatttttgacaagaatagttttgttgagacactggaaggctgggcaaggacagttgtgacaggaagggcaaagctcggaggaatccctgttggagtaatagctgttgaaactcaaactgtgatgcaggtgattcctgctgacccaggacagctcgattctcatgagagggttgttcctcaggctggccaagtatggtttccagattctgcaaccaaaacagctcaagctatattggatttcaacagagaagaacttccacttttcattcttgcatattggaggggcttttcaggtggacaaagggatctttttgaaggtatcctccaggcaggttcaacaatagttgagaatcttaggacatacaaccaacctgtttttgtatacatccccatgatgggtgaacttcgtggtggggcatgggttgtggtggacagtcagatcaattctgaccatatagaaatgtatgctgataggacagccaaaggtaatgtccttgagccagaaggcataattgagatcaaatttagaacaaaagagctgcttgagtccatgggtaggcttgataaacagttgatcacattgaaggcaaaacttcaagaagctaggaatagctggaactttgggatggttgaagacttacaacagcagataaaatctcgtgaaaagcaacttttgcccatatacactcaaatagccaccagatttgcggagcttcatgattcttccctaaggatggctgcaaagggggtgatcagagaaattgtagactgggataaatcccgtgcttacttctataaaaggctacgtaggagaatcgctgagggttcactgatcaagactgtgaaagatgcagctggtgaccagttgtcccataaatctgcaatggacttgatcaaaaactggtttttagattctgatattgcaagaggcaaagaagatgcttgggggaatgatgaagctttctttgcatggaaggatgatcaagggaaatatgaagaaaaactacaagagctacgggttcagaaagtgttggtacaactgacaaacattggtgactccatgtcagatttgaaagctctacctcaaggtcttgctgctcttctaagaaaggtggagccatcgagccgagggcaaataattgaagagcttcgaaaggtcatcagttgatttggtatgtcctttacgagcgaatattcatgctcatacttaggtaacagatattttcaagtgagaaaaagaaatgtatttacaatgctatttgccaaccctatatgcaattgtaatttatcagccaagaggaaaacctcactgtaaattggagaaggttctccaccgatcagttttaatgcttcagtgtaaatttagctttaatcttgggataaactaggagtagattgatattgttaagagtggaaactggccagcattggcagcctatgccatccatggctgttccttggcttgtttagttattatttttgaaataaaaaaaaaaaaaaaaaa

SEQ ID NO: 2 - protein, 2271 aa, J. curcas
MQHVGSTKETTGTGGCCSCNGYINGVVSMRSPATISEVDEFCHALGGNSPIHSILIANNGMAAVKFMRSIRTWAYETFGNEKAILLVAMATPEDMKINAEHIRIADQFVEVPGGTNNNNYANVQLILEMAEGTRVDAVWPGWGHASENPELPDALSAKGIVFLGPPATSMAALGDKIGSSLIAQAADVPTLPWSGSHVKIPPESCLIAIPDEVYREACVYTTEEAIASCQVVGYPAMIKASWGGGGKGIRKVHNDDEVRALFKQVQGEVPGSPIFIMKVASQSRHLEVQLLCDQHGNVAALHSRDCSVQRRHQKIIEEGPITVAPLETVKKLEQAARRLAKSVNYVGAATVEYLYSMETGEYYFLELNPRLQVEHPVTEWIAEVNLPAAQVAVGMGIPLWQIPEIRRFYGVENGGGYDAWRKTSVVATPFDFDKAESTRPKGHCVAVRVTSEDPDDGFKPTSGKVQELSFKSKPNVWAYFSVKSGGGIHEFSDSQFGHVFAFGESRALAIANMVLGLKEIQIRGEIRTNVDYSIDLLHASDYRDNKIHTGWLDSRIAMRVRAKRPPWYLSVVGGALYKASASSAAMVSDYVGYLEKGQIPPKHISLVNSQVSLNIEGSKYVINMVRGGPGSYRLRMNESEIEAEIHTLRDGGLLMQLDGNSHVIYAEEEAAGTRLLIDGRTCLLQNDHDPSKLVAETPCKLLRFLVLDGSHIEADTPYAEVEVMKMCMPLLSPASGVLQFKMSEGQAMQAGELIARLELDDPSAVRKPELFHGSFPILGPPTAISGKVHQRCAASLNAACMILAGYEHNIDEVVQNLLNCLDSPELPFLQWQECLSVLATRLPKDLRNELESKYRGFEGISSSQNVDFPAKLLRGVLEAHLSSCPEKEKGAQERLVEPLMSLVKSYEGGRESHARVIVQSLFDEYLSVEELFRDNIQADVIERLRLQYKKDLLKVVDIVLSHQGVRSKNKLILRLMEQLVYPNPAAYRDKLIRFSQLNHTSYSELALKASQLLEQTKLSELRSIIARSLSELEMFTEDGENMDTPKRKSAINERMEDLVSAPLAVEDALVGLFDHSDHTLQRRVVETYVRRLYQPYLVKESVRMQWHRSGLIASWEFLEEHIGRKNGYEDQMSDEPVMEKHCDRKWGAMVIIKSLQFLPAIISAALRETTHNLHEAIPNRSTELDNYGNMMHIALVGINNQMSLLQDSGDEDQAQERIKKLAKILKEQEVGSSLRTAGVEVISCIIQRDEGRAPMRHSFHWSEEKLYYEEEPLLRHLEPPLSIYLELDKLKSYGNIQYTPSRDRQWHLYTVVDKPVSIQRMFLRTLVRQPTTNEVFTACQGLGMEAPQAQWTMSFTSRSILRSLVAAMEELELNMHNATVKSDHAHMYLCILREQQIDDLVPYPKRVDIEAGQEEVAIGRILEELAREIHASVGVKMHRLNVCEWEVKLWMTSCGQANGAWRVVITNVTGHTCAVHTYRELEDASKHGVVYHSVSVQGPLHGVLVNAVYQSLGVLDRKRLLARRSNTTYCYDFPLAFETALEQIWASQFTGTGKLKCNVLVKATELVFSDQKGSWGTPLVPVDRPAGLNDIGMIAWTMELSTPEFPSGRTILIVANDVTFKAGSFGPREDAFFYAVTDLACTKKLPLIYLAANSGARIGVAEEVKSCFKVGWSDETSPEGGFQYVYLSPEDYTHIASSVIAHELKLSNGETRWVIDAIVGKEDGLGVENLSGSGAIASAYSRAYKETFTLTYVTGRTVGIGAYLARLGMRCMQRVDQPIILTGFSALNKLLGREVYSSHIQLGGPKVMATNGVVHLTVSDDLEGVSAILNWLSCIPPCIGGTLPILGPSDPTERPVEYFPENSCDPRAAISGSLDGNGKWLGGIFDKNSFVETLEGWARTVVTGRAKLGGIPVGVIAVETQTVMQVIPADPGQLDSHERVVPQAGQVWFPDSATKTAQAILDFNREELPLFILAYWRGFSGGQRDLFEGILQAGSTIVENLRTYNQPVFVYIPMMGELRGGAWVVVDSQINSDHIEMYADRTAKGNVLEPEGIIEIKFRTKELLESMGRLDKQLITLKAKLQEARNSWNFGMVEDLQQQIKSREKQLLPIYTQIATRFAELHDSSLRMAAKGVIREIVDWDKSRAYFYKRLRRRIAEGSLIKTVKDAAGDQLSHKSAMDLIKNWFLDSDIARGKEDAWGNDEAFFAWKDDQGKYEEKLQELRVQKVLVQLTNIGDSMSDLKALPQGLAALLRKVEPSSRGQIIEELRKVIS

SEQ ID NO: 3 - DNA, 899 bp, J. curcas
gggcgaattgggcccgacgtcgcatgctcccggccgccatggcggccgcgggaattcgattgaggtgtatagctttcagatgcaacttggtggacccatagttatggcaaccaatggagtagttcatcttactgtctcagatgatctagaaggtgtatctgctatcttgaactggctaagttgtatccctccttgtattggtggcacacttccaattttaggtccttcggatcctactgaaaggcctgtggagtatttcccagaaaactcatgtgatccacgtgctgctatttctggttctttggatggtaatgggaagtggcttgggggcatttttgacaagaatagttttgttgagacactggaaggctgggcaaggacagttgtgacaggaagggcaaagctcggaggaatcctgttggagtaatagctgttgaaactcaaactgtgatgcaggtgattcctgctgacccaggacagctcgattctcatgagagggttgttcctcaggctggccaagtatggtttccagattctgcaaccaaaacagctcaagctatattggatttcaacagagaagaacttccacttttcattcttgctaattggaggggaggagtaagcaagctcggaggaatccctgttggagtattagctgttagaaactcaaactgtgatgcaggtgattcctgctgacccaggacagctcgattctcatagagagggttgttcctcaggctggccaagtatggtttccagattctgcaaccaaaacagctcaagctatattaggatttcaacagagaagaacttccacttttcattcttgctaattggaggggcttttcaggtggacaaagggatctttttgaaggtatcctccaggcaggttcaacaat

SEQ ID NO: 4 - DNA, 899 bp, J. curcas
attgttgaacctgcctggaggataccttcaaaaagatccctttgtccacctgaaaagcccctccaattagcaagaatgaaaagtggaagttcttctctgttgaaatcctaatatagcttgagctgttttggttgcagaatctggaaaccatacttggccagcctgaggaacaaccctctctatgagaatcgagctgtcctgggtcagcaggaatcacctgcatcacagtttgagtttctaacagctaatactccaacagggattcctccgagcttgcttactcctcccctccaattagcaagaatgaaaagtggaagttcttctctgttgaaatccaatatagcttgagctgttttggttgcagaatctggaaaccatacttggccagcctgaggaacaaccctctcatgagaatcgagctgtcctgggtcagcaggaatcacctgcatcacagtttgagtttcaacagctattactccaacaggattcctccgagctttgcccttcctgtcacaactgtccttgcccagccttccagtgtctcaacaaaactattcttgtcaaaaatgcccccaagccacttcccattaccatccaaagaaccagaaatagcagcacgtggatcacatgagttttctgggaaatactccacaggcctttcagtaggatccgaaggacctaaaattggaagtgtgccaccaatacaaggagggatacaacttagccagttcaagatagcagatacaccttctagatcatctgagacagtaagatgaactactccattggttgccataactatgggtccaccaagttgcatctgaaagctatacacctcaatcgaattcccgcggccgccatggcggccgggagcatgcgacgtcgggcccaattcgccc

SEQ ID NO: 5 - DNA, 626 bp, J. curcas
tttgtatacatccccaggatgggtgaacttcgtggtgggggcatgggtggtggtggacagtcagatcaattctgaccatatagaaagtatgctgataggacagccaaaggtaatgtccttgagccagaaggcataattgagatcaaatttagaacaaaagagctgcttgagtccatgggtaggcttgatcagcasttgatcacattgaaggcaaaacttcaagaagctaggaatactggaacttatgggatggttgaagacttacaacagcagataaaatctcgtgaaaagcaacttttgcccatatacactcaaatagccaccagatttgcggagcttcatgattcttccctaaggatggctgcaaagggggtgatcagagaaattgtagactgggataaatcccgtgcttacttctataaaaggctacgtaggagaatcgctgaggaatcactgatcaagactgtgaaagatgcagctggtgaccagttgtcccataaatctgcaatggacttgatcaaaaactggtttttagattctgatattgcaagaggcaaagaagatgcttgggggaatgatgaagctttctttgcatggaaggataatcaagggaaatatgagaagaa

SEQ ID NO: 6 - DNA, 625 bp, J. curcas
ttcttctcatatttcccttgattatccttccatgcaaagaaagcttcatcattcccccaagcatcttctttgcctcttgcaatatcagaatctaaaaaccagtttttgatcaagtccattgcagatttatgggacaactggtcaccagctgcatctttcacagtcttgatcagtgattcctcagcgattctcctacgtagccttttatagaagtaagcacgggatttatcccagtctacaatttctctgatcaccccctttgcagccatccttagggaagaatcatgaagctccgcaaatctggtggctatttgagtgtatatgggcaaaagttgcttttcacgagattttatctgctgttgtaagtcttcaaccatcccataagttccagtattcctagcttcttgaagttttgccttcaatgtgatcaatgctgatcaagcctacccatggactcaagcagctcttttgttctaaatttgatctcaattatgccttctggctcaaggacattacctttggctgtcctatcagcatactttctatatggtcagaattgatctgactgtccaccaccacccatgcccccaccacgaagttcacccatcctggggatgtatacaaa

SEQ ID NO: 7 - DNA, 626 bp, J. cucas; identical to SEQ ID NO: 5

SEQ ID NO: 8 - DNA, 625 bp, J. curcas; identical to SEQ ID NO: 6

SEQ ID NO: 9 - protein, 199 aa, J. curcas
EVYSFQMQLGGPIVMATHGVVHLTVSDDLEGVSAILNWLSCIPPCIGGTLPILGPSDPTERPVEYFPENSCDPRAAISGSLDGNGKWLGGIFDKNSFVETLEGWARTVVTGRAKLGGIPVGVIAVETQTVMQVIPADPGQLDSHERVVPQAGQVWFPDSATKTAQAILDFNREELPLFILANWRGFSGGQRDLFEGILQ

SEQ ID NO: 10 - DNA, 611 bp, J. curcas
gaggtgtacagctctcacatccaacttggtggccccaaagttatggcaaccaatggagtagttcatcttactgtctcagatgatctagaaggtgtatctgctatcttgaactggctaagttgtatccctccttgtattggtggcacacttccaattttaggtccttcggatcctactgaaaggcctgtggagtatttcccagaaaactcatgtgatccacgtgctgctatttctggttctttggatggtaatgggaagtggcttgggggcatttttgacaagaatagttttgttgagacactggaaggctgggcaaggacagttgtgacaggaagggcaaagctcggaggaatccctgttggagtaatagctgttgaaactcaaactgtgatgcaggtgattcctgctgacccaggacagctcgattctcatgagagggttgttcctcaggctggccaagtatggtttccagattctgcaaccaaaacagctcaagctatattggatttcaacagagaagaacttccacttttcattcttgcatattggaggggcttttcaggtggacaaagggatctttttgaaggtatcctccaggcaggttcaacaat

SEQ ID NO: 11 - protein, 203 aa, J. curcas
EVYSSHIQLGGPKVMATNGVVHLTVSDDLEGVSAILNWLSCIPPCIGGTLPILGPSDPTERPVEYFPENSCDPRAAISGSLDGNGKWLGGIFDKNSFVETLEGWARTVVTGRAKLGGIPVGVIAVETQTVMQVIPADPGQLDSHERVVPQAGQVWFPDSATKTAQAILDFNREELPLFILAYWRGFSGGQRDLFEGILQAGST

SEQ ID NO: 12 - DNA, 6634 bp, J. curcas
agtagatgaattctgcaatgcccttggagggaatagtccaattcatagtattttaatagcaaacgatggaatggcagctgtcaagtttatgcgtagtattagaacatgggcttatgaaacatttggcaatgagaaggcaatcttgttggtggccatggcaactccggaagacatgaaaatcaatgcagagcatattagaattgctgatcaatttgtagaagttcctggtgggacaaacaataataactatgccaatgtgcagctgatcttagagatggcagaaggaactcgtgttgatgccgtttggcctggttggggccatgcatctgagaaccctgagctgccagatgcactgagtgcaaaaggtatcgtatttcttgggcccccagctacatctatggctgcactgggtgataaaatcggctcatctttgattgctcaagcagcagatgtccctactcttccatggagtggctctcatgtgaaaattcctccagaaagttgtttgattgccatcccagatgaggtatacagagaagcatgtgtatatacaacagaggaagcgattgcaagttgtcaagttgttggataccctgcaatgatcaaggcatcatggggtggtggtggtaaaggcataagaaaggttcataatgatgatgaagtcagggcattgttcaagcaagttcaaggtgaagttccaggatcacccatatttataatgaaggttgcttcccagagtcgacatttggaagtccagttactctgtgatcagcatgggaatgtagctgctttgcacagccgtgattgcagtgttcagaggcggcaccaaaagataattgaggagggtccaattactgttgcgcctctggagacagtcaaaaagctagaacaagcagctcgaaggttagcgaaaagtgtgaattatgttggagcagctactgttgagtatttgtacagtatggaaactggagaatactactttttagaactcaatcctcggttacaggtggagcacccagtgactgagtggattgctgaagtaaatttgccagctgcccaggtagctgttgggatgggaattcctctctggcaaattcctgagataaggcgattttatggagtggaaaatggtggaggatatgatgcttggaggaaaacttcagtggttgctactccttttgattttgacaaggctgagtctactaggccaaaaggccattgtgtggctgtgcgtgtgacaagtgaggatccagatgatggttttaagcctacaagtggaaaagtacaggagctaagttttaaaagcaagccaaatgtgtgggcttatttctctgttaagtctggtggaggcattcatgaattttcagattctcaatttggtcatgtttttgcgtttggagaatccagagctttggctatagcaaatatggtccttgggctgaaagaaattcaaattcgaggagaaattcggactaatgttgactactcaattgatcttttacacgcttctgactatagggacaacaaaatccacacaggttggttggacagtagaattgcaatgcgggttagagcaaaaaggcccccttggtacctctctgttgttggaggggctttatacaaagcatctgctagcagtgcagctatggtttcagattatgttggttaccttgaaaaggggcaaatccctcctaagcacatatcacttgttaactctcaagtttcattgaacattgaaggaagcaaatacgtgataaacatggttagaggggggccaggaagctatagattgagaatgaatgaatcagagattgaagcagagatacatactttacgtgatggaggtttattgatgcagttggatggaaacagtcatgtgatatatgcagaagaagaagcagctggaactcgtcttcttattgatggaaggacttgcttgctgcagaatgatcacgatccttcaaagttagtggcagaaacgccatgcaagctgctgaggtttttggttttggatggtagtcatattgaagctgatactccatatgcggaggttgaggtcatgaagatgtgcatgcctctcctttcacctgcttctggagttcttcagtttaaaatgtctgaaggtcaagcaatgcaggctggtgagcttatagcacggcttgaacttgatgatccttcggctgtacgaaagcctgaactttttcatgggagcttcccaatactggggccaccaactgctatttctggtaaagttcatcagagatgtgctgcaagtctgaatgcagcttgcatgattcttgctggctatgaacacaatattgatgaagtagtacaaaacttgctaaactgtctagacagtcctgaactacctttccttcagtggcaagagtgcttgtctgttctggcaactcgccttcccaaagatcttagaaatgagttggaatcaaaatacagggggtttgaagggatttcgagctcccagaatgttgacttccctgccaaattgttaaggggtgttcttgaggcccatctatcctcctgtcctgaaaaagaaaaaggtgcacaagaaaggcttgttgaacctttgatgagtcttgtaaagtcttatgagggaggacgggagagtcatgcccgcgtcattgttcagtcactttttgacgagtatttatctgttgaagaattgttcagagataacatccaggctgatgtgattgaacgtcttagactccaatacaagaaagatctgttgaaggttgttgacattgtcctttctcatcagggtgtgaggagtaaaaataagctgatattgcggcttatggaacaattggtttatcctaaccctgctgcatatagggataaactgatccgcttctctcaacttaaccatacaagttattctgagttggcactgaaggcaagtcaactgctagaacaaaccaaactaagtgaacttcgttccatcattgctagaagcctctctgaattggagatgtttactgaggatggtgaaaatatggatactcctaagaggaaaagtgccattaatgaacgaatggaggatctagtgagcgctcctttggctgttgaggatgctcttgtggggctgtttgatcacagtgatcacactcttcagaggcgggtggtggaaacctatgttcgaaggctataccagccatatcttgtaaaggagagtgtcaggatgcagtggcatagatctggtctgattgcttcatgggagttcttggaagaacatattggaagaaagaatggctatgaagatcaaatgtctgatgaaccagtaatggagaaacactgtgacaggaaatggggagccatggttattatcaaatctctacagtttttacctgcaattattagtgctgcactaagagaaacgacccacaatcttcatgaagccattccaaatagatctacagaactagataactatggtaatatgatgcatattgctttggtgggcatcaacaaccagatgagtctacttcaggatagtggtgatgaggatcaggctcaagagagaattaaaaagttagcaaaaattcttaaagaacaagaagtaggctccagtttgcgcaccgcaggtgttgaagttattagctgcatcatacaaagggatgaaggaagggcccctatgagacactcctttcactggtcagaagaaaagctctactatgaggaagaacctctattgcgacatctagaacctccactgtccatctatctagaattggataaacttaaaagttatgggaacatacagtacactccatcacgggacagacagtggcacttgtacactgttgtagacaagccagtgtcaatccagaggatgtttcttagaacccttgtgaggcaacctacaacaaatgaagtgttcaccgcatgtcaaggactgggcatggaagcacctcaagcacaatggactatgtcctttacttcaagaagcattttgaggtccttagtggctgcgatggaggagttggaacttaatatgcataatgctactgtcaaatctgaccatgctcatatgtatctctgtattttgcgggagcaacaaatagatgatcttgtgccataccccaagagagttgatattgaggctggccaggaagaagttgcaattggccgaatcttggaagaactggctagggaaatacatgcatccgttggtgtgaaaatgcataggttaaatgtttgtgaatgggaagtgaagctctggatgacatcatgtggacaggcaaatggtgcttggcgagttgttatcactaatgtaactggtcacacctgtgctgtacatacataccgggaactagaggatgccagcaaacatggagtggtgtaccattcagtctctgtacagggtcctctgcatggtgtattggtaaatgcagtttatcagtccttgggagttcttgatcgaaaacgtcttttggcaaggagaagcaacaccacatactgctacgattttccactggcatttgagacagccttggaacaaatatgggcatcccagtttactggaactggaaaactgaagtgtaatgttcttgtcaaagccacagagcttgtattttctgatcagaaaggcagctggggtactcctcttgttcctgtggatcgcccagctgggctcaatgacattggcatgatagcatggaccatggaattgtctacccctgagtttccttctggaaggacaattttgatagtagcaaatgatgtcaccttcaaagctgggtcttttggcccaagagaggatgcattcttctatgctgtaaccgatcttgcttgcacaaaaaagcttccattaatttatttggcagcaaattctggtgcccgaattggggttgccgaggaagtgaaatcctgttttaaagttggttggtcagatgaaacatcccctgagggtggttttcaatatgtatatttgagtcctgaagattacactcacattgcatcatctgtcatagcacatgagttgaagctatctaatggagaaaccagatgggtgatagatgccattgttggaaaggaggatggcttgggggtagagaacttatctggaagtggggccattgctagtgcatattctagggcatacaaagaaacttttaccttaacatatgtcacaggtagaacagtgggaattggagcttacctagctcggcttgggatgcgatgcatgcaaagggttgatcagcccattattttgactggtttctctgcattgaacaaacttcttggtcgtgaggtgtacagctctcacatccaacttggtggccccaaagttatggcaaccaatggagtagttcatcttactgtctcagatgatctagaaggtgtatctgctatcttgaactggctaagttgtatccctccttgtattggtggcacacttccaattttaggtccttcggatcctactgaaaggcctgtggagtatttcccagaaaactcatgtgatccacgtgctgctatttctggttctttggatggtaatgggaagtggcttgggggcatttttgacaagaatagttttgttgagacactggaaggctgggcaaggacagttgtgacaggaagggcaaagctcggaggaatccctgttggagtaatagctgttgaaactcaaactgtgatgcaggtgattcctgctgacccaggacagctcgattctcatgagagggttgttcctcaggctggccaagtatggtttccagattctgcaaccaaaacagctcaagctatattggatttcaacagagaagaacttccacttttcattcttgcatattggaggggcttttcaggtggacaaagggatctttttgaaggtatcctccaggcaggttcaacaatagttgagaatcttaggacatacaaccaacctgtttttgtatacatccccatgatgggtgaacttcgtggtggggcatgggttgtggtggacagtcagatcaattctgaccatatagaaatgtatgctgataggacagccaaaggtaatgtccttgagccagaaggcataattgagatcaaatttagaacaaaagagctgcttgagtccatgggtaggcttgataaacagttgatcacattgaaggcaaaacttcaagaagctaggaatagctggaactttgggatggttgaagacttacaacagcagataaaatctcgtgaaaagcaacttttgcccatatacactcaaatagccaccagatttgcggagcttcatgattcttccctaaggatggctgcaaagggggtgatcagagaaattgtagactgggataaatcccgtgcttacttctataaaaggctacgtaggagaatcgctgagggttcactgatcaagactgtgaaagatgcagctggtgaccagttgtcccataaatctgcaatggacttgatcaaaaactggtttttagattctgatattgcaagaggcaaagaagatgcttgggggaatgatgaagctttctttgcatggaaggatgatcaagggaaatatgaagaaaaactacaagagctacgggttcagaaagtgttggtacaactgacaaacattggtgactccatgtcagatttgaaagctctacctcaaggtcttgct

SEQ ID NO: 13 - protein, 2211 aa, J. curcas
VDEFCNALGGNSPIHSILIANDGMAAVKFMRSIRTWAYETFGNEKAILLVAMATPEDMKINAEHIRIADQFVEVPGGTNNNNYANVQLILEMAEGTRVDAVWPGWGHASENPELPDALSAKGIVFLGPPATSMAALGDKIGSSLIAQAADVPTLPWSGSHVKIPPESCLIAIPDEVYREACVYTTEEAIASCQVVGYPAMIKASWGGGGKGIRKVHNDDEVRALFKQVQGEVPGSPIFIMKVASQSRHLEVQLLCDQHGNVAALHSRDCSVQRRHQKIIEEGPITVAPLETVKKLEQAARRLAKSVNYVGAATVEYLYSMETGEYYFLELNPRLQVEHPVTEWIAEVNLPAAQVAVGMGIPLWQIPEIRRFYGVENGGGYDAWRKTSVVATPFDFDKAESTRPKGHCVAVRVTSEDPDDGFKPTSGKVQELSFKSKPNVWAYFSVKSGGGIHEFSDSQFGHVFAFGESRALAIANMVLGLKEIQIRGEIRTNVDYSIDLLHASDYRDNKIHTGWLDSRIAMRVRAKRPPWYLSVVGGALYKASASSAAMVSDYVGYLEKGQIPPKHISLVNSQVSLNIEGSKYVINMVRGGPGSYRLRMNESEIEAEIHTLRDGGLLMQLDGNSHVIYAEEEAAGTRLLIDGRTCLLQNDHDPSKLVAETPCKLLRFLVLDGSHIEADTPYAEVEVMKMCMPLLSPASGVLQFKMSEGQAMQAGELIARLELDDPSAVRKPELFHGSFPILGPPTAISGKVHQRCAASLNAACMILAGYEHNIDEVVQNLLNCLDSPELPFLQWQECLSVLATRLPKDLRNELESKYRGFEGISSSQNVDFPAKLLRGVLEAHLSSCPEKEKGAQERLVEPLMSLVKSYEGGRESHARVIVQSLFDEYLSVEELFRDNIQADVIERLRLQYKKDLLKVVDIVLSHQGVRSKNKLILRLMEQLVYPNPAAYRDKLIRFSQLNHTSYSELALKASQLLEQTKLSELRSIIARSLSELEMFTEDGENMDTPKRKSAINERMEDLVSAPLAVEDALVGLFDHSDHTLQRRVVETYVRRLYQPYLVKESVRMQWHRSGLIASWEFLEEHIGRKNGYEDQMSDEPVMEKHCDRKWGAMVIIKSLQFLPAIISAALRETTHNLHEAIPNRSTELDNYGNMMHIALVGINNQMSLLQDSGDEDQAQERIKKLAKILKEQEVGSSLRTAGVEVISCIIQRDEGRAPMRHSFHWSEEKLYYEEEPLLRHLEPPLSIYLELDKLKSYGNIQYTPSRDRQWHLYTVVDKPVSIQRMFLRTLVRQPTTNEVFTACQGLGMEAPQAQWTMSFTSRSILRSLVAAMEELELNMHNATVKSDHAHMYLCILREQQIDDLVPYPKRVDIEAGQEEVAIGRILEELAREIHASVGVKMHRLNVCEWEVKLWMTSCGQANGAWRVVITNVTGHTCAVHTYRELEDASKHGVVYHSVSVQGPLHGVLVNAVYQSLGVLDRKRLLARRSNTTYCYDFPLAFETALEQIWASQFTGTGKLKCNVLVKATELVFSDQKGSWGTPLVPVDRPAGLNDIGMIAWTMELSTPEFPSGRTILIVANDVTFKAGSFGPREDAFFYAVTDLACTKKLPLIYLAANSGARIGVAEEVKSCFKVGWSDETSPEGGFQYVYLSPEDYTHIASSVIAHELKLSNGETRWVIDAIVGKEDGLGVENLSGSGAIASAYSRAYKETFTLTYVTGRTVGIGAYLARLGMRCMQRVDQPIILTGFSALNKLLGREVYSSHIQLGGPKVMATNGVVHLTVSDDLEGVSAILNWLSCIPPCIGGTLPILGPSDPTERPVEYFPENSCDPRAAISGSLDGNGKWLGGIFDKNSFVETLEGWARTVVTGRAKLGGIPVGVIAVETQTVMQVIPADPGQLDSHERVVPQAGQVWFPDSATKTAQAILDFNREELPLFILAYWRGFSGGQRDLFEGILQAGSTIVENLRTYNQPVFVYIPMMGELRGGAWVVVDSQINSDHIEMYADRTAKGNVLEPEGIIEIKFRTKELLESMGRLDKQLITLKAKLQEARNSWNFGMVEDLQQQIKSREKQLLPIYTQIATRFAELHDSSLRMAAKGVIREIVDWDKSRAYFYKRLRRRIAEGSLIKTVKDAAGDQLSHKSAMDLIKNWFLDSDIARGKEDAWGNDEAFFAWKDDQGKYEEKLQELRVQKVLVQLTNIGDSMSDLKALPQGLA

SEQ ID NO: 14 - protein, 11358 aa, J. curcas
ATTTAATGCTGTTGCTCTATGTTTTAATGTATAGTGGATAAATGGATATGGCAGTATGCTGTGCTCTATCTGGTAGCTAGGAGATTTTTTTATTTGATGATGACAATTACAATAATTTTTTTAAAAGTTTTTATCTATTTTGGTGATGATAAATCCATGATGATAATGAAAAATGTTAATATGATACTCCTTAGGCTGCTATTTCATGCATTCTCTCCATGACTCTTGCATATTTAATGCTGTTGCACTATGTTTTAATCTGTGGTGGACAAATGGATATGGTCGCATACTGTGCTATCTGGTAGCTAGGAAGTAAACATTTTTCCTATTTGATGATGACAATTACAGTGACCTTTTTTTTTTTTTAAAAGTTCTTACCTTTTTGGGGAAGAAAACCCCCGGTGATAATGAAAAATGTTGGAAGAGGCAAACTCCCACAAAAGAGTGGTTTCTCACCTCATTTTCCTTAACTTCCAATTCTATCTTAGTCATTAATGAGTTGCCAATCCACTGTTTCATATTCTTTTTCTTTTTAGTATATTTTTCTAAGACTTAATTAATTTTGATGTAGGTGGAGCACCCAGTGACTGAGTGGATTGCTGAAGTAAATTTGCCAGCTGCCCAGGTAGCTGTTGGGATGGGAATTCCTCTCTGGCAAATTCCTGGTATGGCATAAAAAAGATTTTGAAATTTGAAATTAGCTGTCATTAGTCATGGGGCATATGTTTTTCTGATAATATGTGTCAATTTCCTTTGTTTAATTAGAGATAAGGCGATTTTATGGAGTGGAAAATGGTGGAGGATATGATGCTTGGAGGAAAACTTCAGTGGTTGCTACTCCTTTTGATTTTGACAAGGCTGAGTCTACTAGGCCAAAAGGCCATTGTGTGGCTGTGCGTGTGACAAGTGAGGATCCAGATGATGGTTTTAAGCCTACAAGTGGAAAAGTACAGGTACGGCTTTCAGGTCTAGAATTCTTATTTGAAAGTTTTCTTCTCCTTATAATGAATCTATATTGAAATTGCTTTGTACAGGAGCTAAGTTTTAAAAGCAAGCCAAATGTGTGGGCTTATTTCTCTGTTAAGGTGCTTTTTCTATTTTCCCTTGGTTTCTTTTCCCAAAAGAATAATTTTCTGTCAGCAGATCCTTGGATTTGTAAGATTAATTTCTAAGTTTGATCCCCGTTGTTCCCACCGCAAATATATGCTCCGCTTATTTTCTTAGAGGTAGGGAATGGAATTCGGATCTGTGTTCTTGTGGCAGGGTTCTAGCAAGCATGGTGCGTGCATGGATCAAAGGTTAGCTCATGAAGTAAGTACGCTCAGTCGATTTATTGAAGTTTTTCCTTAAGATCTTATTGAATGCTGAAGAGTAGTTAAAGCAGCAATGTGGCTATTTTTGTAAGGGCTTTTTATTTCCTGGTTCTTTCCAAAAATAATTTTCTTCAGCAGAACTGGATTCTAAGATTAATGCTTAAGTTCTGGTGCACAGTCTGGTGGAGGCATTCATGAATTTTCAGATTCTCAATTTGGTAAGTCGCAGCACTCTTTTTTAAATTTGTTGCACTAATTGAAATTGATACATTTAGTGCAACCTGATGAGAAGCATCCTTTTTTCCTATCTTTTGTAATTTGAGTTGTTTGATAATCACAATAGTAAATTTAAACTAACACTGTATGATTGCTTTTTTTGATTGCCAAAATGGTTTAGCTATCCGTCTTTCTGTGTGTTGAAAGTGAAGGAAAAATTATTGTTTTAATGCTATTTGTGTTTGGAAAATGTCTACCTTATCCGGGTCCATAGTTATTTGTTGAGAGCTGTGTGCTTTAACTATTAGCAGCAAGTTGATAGCATTGATGAAACCATGAGAATTTGACTCTTTCAGTTAAGTGAAAGGTTATGCTTCTGCTGACTCCAACATATGCAGTACACATCCCTTCTTGAACAGCATTTAACTCTGCATAGGACCTTATGGCAGGAGAAGATTTAAAAATTTTTTGATGCTGTATAGAATCACCCGCAGATTGATCAATATCCTCTTTAATCTATGAGTGAGCACTGTTATTGGATTAGTTTTCCAATCATTTACATATTGCTAACCTTCATATTTTCTATTTATAAATGTTTTTTCTGACAATGATTCATGTCAACATTCTTTAACCTATAACAGTGGATGTTTTCCATCAAGTTTTCTGTAGATGTATCTTTGAAATGTAATTCTTTTGTTGCGATGATCTATGCTGCTCAAGTTTGTTATGATCTTTTTCTTAGGACATGTTTTTGCGTTTGGAGAATCCAGAGCTTTGGCTATAGCAAATATGGTCCTTGGGCTGAAAGAAATTCAAATTCGAGGAGAAATTCGGACTAATGTTGACTACTCAATTGATCTTTTACACGTAATAATCCATTTCTGGTTACCTTCTTTTTTCACATATTAAGAGAGAAAATTAAAAAGATCCCTCGGGAACAAAAATTAACCTGAGATGAAATGTGTTATCCAGGCTTCTGACTATAGGGACAACAAAATCCACACAGGTTGGTTGGACAGTAGAATTGCAATGCGGGTTAGAGCAAAAAGGCCCCCTTGGTACCTCTCTGTTGTTGGAGGGGCTTTATACGTAAGTAGTCATGCATTGGTGAAAGAGGAATAGTTTCCTTTTATTCTGCTATATTCTATTCTACAGAATTCACCACTTATGGTTCCTGTTTTGTTTGCCTCAGAAAGCATCTGCTAGCAGTGCAGCTATGGTTTCAGATTATGTTGGTTACCTTGAAAAGGGGCAAATCCCTCCTAAGGTACTTAATGAAGAATTCATTTTTATGAAAATTTATTGTGTATTCAGATGGTTTTTAGTAACATTGATGTCAATCAATTCTGATTTCAGCACATATCACTTGTTAACTCTCAAGTTTCATTGAACATTGAAGGAAGCAAATACGTGGTATGAATACCTATCAGTATGATTATTCCTCTGAAATGTCAAATTTTCTGTGTATGTTTTTATAATCTAGAAGAGCTGGTTCATAGGCTTTGATAATACATGCCATTAACACTTTGAAAAATTAATCCTAGATAAACATGGTTAGAGGGGGGCCAGGAAGCTATAGATTGAGAATGAATGAATCAGAGATTGAAGCAGAGATACATACTTTACGTGATGGAGGTTTATTGATGCAGGCAAGCATTTATTTCTCCATAATACTTTTGTTATTAATTCTTCATGGATGTTCAGCACTTGTGTTATGTTGCTTTATGATTGGCATCTTTTTTTTTTTCTTTTTTTCACTATTATTCTTCTTCCTCTTCTTTCATGACCTTATTTCTGGACTTGATAATGGAATCTGTTACTTTTATTTTGTTACAGTTGGATGGAAACAGTCATGTGATATATGCAGAAGAAGAAGCAGCTGGAACTCGTCTTCTTATTGATGGAAGGACTTGCTTGCTGCAGAATGATCACGATCCTTCAAAGTTAGTGGCAGAAACGCCATGCAAGCTGCTGAGGTTTTTGGTTTTGGATGGTAGTCATATTGAAGCTGATACTCCATATGCGGAGGTTGAGGTCATGAAGATGTGCATGCCTCTCCTTTCACCTGCTTCTGGAGTTCTTCAGTTTAAAATGTCTGAAGGTCAAGCAATGCAGGTATGTCAAATTTCACAGGATATATATATACTTACAGAGTTTTAGTATGAAGTTAAAGAATCTGTTTTCCTCTTAGGCTGGTGAGCTTATAGCACGGCTTGAACTTGATGATCCTTCGGCTGTACGAAAGCCTGAACTTTTTCATGGGAGCTTCCCAATACTGGGGCCACCAACTGCTATTTCTGGTAAAGTTCATCAGAGATGTGCTGCAAGTCTGAATGCAGCTTGCATGATTCTTGCTGGCTATGAACACAATATTGATGAAGTAAGTATTCTCCTGTGTCCTATGGATACTATAGAGTTTAAAATCCTTATCATGAATCAATAGTATGCTCTTACCTTTTGTTGCTGTGTGCATGGACTTTAAAGATGTTGCAAAAATTGTTTTTCTGTTATATTAACAACTATTTGTTGGAAACTAATGTTCTATCTGAACAATTTTGATAGGTAGTACAAAACTTGCTAAACTGTCTAGACAGTCCTGAACTACCTTTCCTTCAGTGGCAAGAGTGCTTGTCTGTTCTGGCAACTCGCCTTCCCAAAGATCTTAGAAATGAGGTGAATGTTTATCATGTCTTTGATCTCTTCTTTTGTGACCATAAAGTGGCTTTAGGCTTACAATATGATCTGATGTTGCCTTGGCCCAAACATTACAGTTGGAATCAAAATACAGGGGGTTTGAAGGGATTTCGAGCTCCCAAAATGTTGACTTCCCTGCCAAATTGTTAAGGGGTGTTCTTGAGTGTGTTCCTTATTTTGAAACCCTTTCTCACTTTTCTTTGAAGAAAATGAAGAAATTGAAAGTTTGCATATACAGACCTATGCAAGTCCGGGATGCCCATCTTCCCAAACATCCTTACTGATAATGGTATGTTTTTGTGTCTCTGATCTCCCCTTGTCCACCCATAAAGTGACTTTAAGCTTATAATCCTTACTGGGAAAACCTTGGCCCCTGTTTTTGTTTGAACCCTGCATACAACGGGACTGAATTAATTCGATTTGCCCATAATGTTGACTTCCCTGCCAATGTGTTAAACAACTTCCTTGAGGTGTGTTCCTTAATTTGAACCCTTTTCTCGCTTTTCTTGAAAGAAACATTGCATGTAATCCCTACTTGCTGCTTGCTTTTTCTCCACGCCCTGTTATCCTCCTGTCCTGATAAACATATTGGTGCTCCTCAACGGCTTGTAGAACCTTTACACGATTTATATTGCGTCTATGAAGCATCAAGATCAAGTAGTCTTGCCCGTGTCATTGTTCAATCACTTTTTGACGAGTATTTATCTGTTGAAGAATTGTTCAGAGATAACATCCAGGTAAGCATGCATGCATCAAATATATCAATGGGTGTTGTAATGCCAGTATCTCATTATAATTATTTGAGATTGACAAACTTTTTTCTAATTTTTTGTCGATCGCAGATACCATAGTATTAAATCATGACATTGAATGCTATTTTCTTGTCTTTTAGTGGTGACCTTACCATTTATTTGCTTGTATGAACCTGTATTCTTCTGCATACAATCTACTTGTCGTACTTATTTGTCTCTAACTGCGGATAAGATCGGGCCATAAATTCAATTTCCTTGATGAATGGTGAACAAGCTTGTCTGAGCCCTGTATATTTTAGTCACAGTTCGATAATTTTTACTTTCAGGCTAAAATAATAGGAATTTGGTGGATGACTCCACAATTTGTTACTACATTAGAGCGGCTCATCACTTTCTAATTTTATATTTTATATTTTTTATTTAGGCTGATGTGATTGAACGTCTTAGACTCCAATACAAGAAAGATCTGTTGAAGGTTGTTGACATTGTCCTTTCTCATCAGGTATCAAATTTATAATTGTGCCCTTTCTTTCTTTTTTTTTTTTTTTTTTTTTTCAAATTCGATTTTTGATGTTTACATTTATGCATTATATACTGTGTCGTTCTCTTATAGGGTGTGAGGAGTAAAAATAAGCTGATATTGCGGCTTATGGAACAATTGGTTTATCCTAACCCTGCTGCATATAGGGATAAACTGATCCGCTTCTCTCAACTTAACCATACAAGTTATTCTGAGGTTGGTTCTTACACTTGATGAAGTTGTACTTAACATGATCTATGTTTCTTTTTTGACTATCAAATCATTTTAATTTTGTACTGTTTGTTGCAGGGTTGCAACAAAAACAGTATTCTAATGTTTTCCCTTTAAGGATTATTTCTTCTGTCTCACTTTTTTTGTGGTCATTGGTTAATGCGATTTCTCAATTCAATATCATTATCAGTAAGTTTGTTTTTCAAAATCGTCATACCTGATTGCATATGTCACTTATAGTTTGAGAGCATTGAGCTGGCTGCGATCGATATGACATTCAGGCTTTATTTTGGCGGTAGGTGGCCCCAAGTCGAGGTATCTTCCCCGTAATAATGCCTCTCTTTTTTTTTCATCATGGTCATAGCATACGTCTTTTTAGGGAGTAAAAAGGATTCGCTAGACATGTTTCTACTGTGCATTAGAAACGTCGCTCTTCACTACATCAGTATTTGAGGTGTCTACACTGATGAGTGACTAACATGATCTATGTTCTTTTTGACTATCAATCATTTATTTGTACTGTTGTGCAGGGTGCACAAAAACAGTATCTAATGTTTTCCCTTTAAGGATTATTTCTTCTGTCTCACTTTTTTGTGGTCATGGTTAATGCGATTCTCAATTCAATATCATTATCAGTAAGTTGTTTTCAAAATCTTCATACCTGATTTGCATATGCAGTTGGCACTGAAGGCAAGTCAACTGCTAGAACAAACCAAACTAAGTGAACTTCGTTCCATCATTGCTAGAAGCCTCTCTGAATTGGAGATGTTTACTGAGGATGGTGAAAATATGGATACTCCTAAGAGGAAAAGTGCCATTAATGAACGAATGGAGGATCTAGTGAGCGCTCCTTTGGCTGTTGAGGATGCTCTTGTGGGGCTGTTTGATCACAGTGATCACACTCTTCAGAGGCGGGTGGTGGAAACCTATGTTCGAAGGCTATACCAGGTACCATATTTTGAACTCTTAAAGGTTTTCTCGATTAAAATTTTACACAATCATTTCTTTAAATTAACTGTTTGTATATGTTATCAGCCATATCTTGTAAAGGAGAGTGTCAGGATGCAGTGGCATAGATCTGGTCTGATTGCTTCATGGGAGTTCTTGGAAGAACATATTGGAAGAAAGAATGGCTATGAAGATCAAATGTCTGATGAACCAGTAATGGAGAAACACTGTGACAGGAAATGGGGAGCCATGGTTATTATCAAATCTCTACAGTTTTTACCTGCAATTATTAGTGCTGCACTAAGAGAAACGACCCACAATCTTCATGAAGCCATTCCAAATAGATCTACAGAACTAGATAACTATGGTAATATGATGCATATTGCTTTGGTGGGCATCAACAACCAGATGAGTCTACTTCAGGATAGGTACTTCCCTCCACTACTATGTAATAACTATTTCTATTAAACCTTGAACATATTGATTTGCACAGCCTGTTAGTAACAATATATTCAGGATCCTATGACTTACAGCTTTTTTTAATAATTTGGAAGCTGTTAAAATTCAGATGATTTGACTATATTTGTAAAAAATTAATGATAGATTTTCAAGAGAATTAAACAATTACACCTTAACACGGACACACACATACAGGAACTTTTTCCAGACCATCAAACTTCTGGTAAAACATCAATATGACCTTGTCCCTTTCTTACTCATGATGTCCTTTTGCAGTGGTGATGAGGATCAGGCTCAAGAGAGAATTAAAAAGTTAGCAAAAATTCTTAAAGAACAAGAAGTAGGCTCCAGTTTGCGCACCGCAGGTGTTGAAGTTATTAGCTGCATCATACAAAGGGATGAAGGAAGGGCCCCTATGAGACACTCCTTTCACTGGTCAGAAGAAAAGCTCTACTATGAGGAAGAACCTCTATTGCGACATCTAGAACCTCCACTGTCCATCTATCTAGAATTGGTTTGTTTCTATACATCTGTTCCCAACTATTTTGTTTTAGGGGGAAAAAAGTAAAGCAGAGAGAAACTTTACATACTTTGATTTGAAGCTTATAGCTATCTGTTTTTGTCCAGGATAAACTTAAAAGTTATGGGAACATACAGTACACTCCATCACGGGACAGACAGTGGCACTTGTACACTGTTGTAGACAAGCCAGTGTCAATCCAGAGGATGTTTCTTAGAACCCTTGTGAGGCAACCTACAACAAATGAAGTGTTCACCGCATGTCAAGGACTGGGCATGGAAGCACCTCAAGCACAATGGACTATGTCCTTTACTTCAAGAAGCATTTTGAGGTCCTTAGTGGCTGCGATGGAGGAGTTGGAACTTAATATGCATAATGCTACTGTCAAATCTGACCATGCTCATATGTATCTCTGTATTTTGCGGGAGCAACAAATAGATGATCTTGTGCCATACCCCAAGTAATTGTTTTAACTTCTGTTCTCTTTCATGTGATAAGCTGGTGAATTTCTGTTATAGGTTCTGATGCTCTATGGTTATCATCAGGAGAGTTGATATTGAGGCTGGCCAGGAAGAAGTTGCAATTGGCCGAATCTTGGAAGAACTGGCTAGGGAAATACATGCATCCGTTGGTGTGAAAATGCATAGGTTAAATGTTTGTGAATGGGAAGTGAAGCTCTGGATGACATCATGTGGACAGGCAAATGGTGCTTGGCGAGTTGTTATCACTAATGTAACTGGTCACACCTGTGCTGTACATGTAAGTATATGCCATCTCATACACGCTCAAGAAAACTTCTTTCATCCATCCATGCATTTAGATTTTGATTTTTGATGCATGAAGAGGAGGTATTTCATCCATTCATGCATTATTCTATCCAGATCTATAACTGATTTAAATAAATGGAGAATTAAAGGAAAAATAATTTTGACGTCAGTTTGAATGGTTTGGAAATTTATGCAAGTATAGCATGGAAAACTTTTCTTGCTGTTGTAAGCAAGCAATTTCATGTGTAATACAAGTGCATGTGAGATGTGAAATTACCATCTCGGAAATTTAGATGGAAAAATATAATATATTGTTCTGGTTCCTGCACTAGTTTAAACTTTTTTATACGGAAAGGATCAGACTTAGAATTATTAGAGAGGTATTGGTAACAGAGGATTCATATAGTCAATCAACTAGTTTGGAACTAAAGCTTAGTTGTTGTTGTTGGAAAGTTCAGATCATTTTCTAATGTTCTGGTTAATATGCAATCAGACATACCGGGAACTAGAGGATGCCAGCAAACATGGAGTGGTGTACCATTCAGTCTCTGTACAGGGTCCTCTGCATGGTGTATTGGTAAATGCAGTTTATCAGTCCTTGGGAGTTCTTGATCGAAAACGTCTTTTGGCAAGGAGAAGCAACACCACATACTGCTACGATTTTCCACTGGTCAGTCTTTTGATATTTACTTATAACATAGTTCGTAAATTAATTTTTTCTTGATCCAAATTATGTTGCTGAATATGTAATACTTTCAGGCATTTGAGACAGCCTTGGAACAAATATGGGCATCCCAGTTTACTGGAACTGGAAAACTGAAGTGTAATGTTCTTGTCAAAGCCACAGAGCTTGTATTTTCTGATCAGAAAGGCAGCTGGGGTACTCCTCTTGTTCCTGTGGATCGCCCAGCTGGGCTCAATGACATTGGCATGATAGCATGGACCATGGAATTGTCTACCCCTGAGTTTCCTTCTGGAAGGACAATTTTGATAGTAGCAAATGATGTCACCTTCAAAGCTGGGTCTTTTGGCCCAAGAGAGGATGCATTCTTCTATGCTGTAACCGATCTTGCTTGCACAAAAAAGCTTCCATTAATTTATTTGGCAGCAAATTCTGGTGCCCGAATTGGGGTTGCCGAGGAAGTGAAATCCTGTTTTAAAGTTGGTTGGTCAGATGAAACATCCCCTGAGGGTGGTTTTCAATATGTATATTTGAGTCCTGAAGATTACACTCACATTGCATCATCTGTCATAGCACATGAGTTGAAGCTATCTAATGGAGAAACCAGATGGGTGATAGATGCCATTGTTGGAAAGGAGGATGGCTTGGGGGTAGAGAACTTATCTGGAAGTGGGGCCATTGCTAGTGCATATTCTAGGGCATACAAAGAAACTTTTACCTTAACATATGTCACAGGTAGAACAGTGGGAATTGGAGCTTACCTAGCTCGGCTTGGGATGCGATGCATGCAAAGGGTTGATCAGCCCATTATTTTGACTGGTTTCTCTGCATTGAACAAACTTCTTGGTCGTGAGGTGTACAGCTCTCACATCCAACTTGGTGGCCCCAAAGTTATGGCAACCAATGGAGTAGTTCATCTTACTGTCTCAGATGATCTAGAAGGTGTATCTGCTATCTTGAACTGGCTAAGTTGTATCCCTCCTTGTATTGGTGGCACACTTCCAATTTTAGGTCCTTCGGATCCTACTGAAAGGCCTGTGGAGTATTTCCCAGAAAACTCATGTGATCCACGTGCTGCTATTTCTGGTTCTTTGGATGGTAATGGGAAGTGGCTTGGGGGCATTTTTGACAAGAATAGTTTTGTTGAGACACTGGAAGGCTGGGCAAGGACAGTTGTGACAGGAAGGGCAAAGCTCGGAGGAATCCCTGTTGGAGTAATAGCTGTTGAAACTCAAACTGTGATGCAGGTGATTCCTGCTGACCCAGGACAGCTCGATTCTCATGAGAGGGTTGTTCCTCAGGCTGGCCAAGTATGGTTTCCAGATTCTGCAACCAAAACAGCTCAAGCTATATTGGATTTCAACAGAGAAGAACTTCCACTTTTCATTCTTGCATATTGGAGGGGCTTTTCAGGTGGACAAAGGGATCTTTTTGAAGGTATCCTCCAGGCAGGTTCAACAATAGTTGAGAATCTTAGGACATACAACCAACCTGTTTTTGTATACATCCCCATGATGGGTGAACTTCGTGGTGGGGCATGGGTTGTGGTGGACAGTCAGATCAATTCTGACCATATAGAAATGTATGCTGATAGGACAGCCAAAGGTAATGTCCTTGAGCCAGAAGGCATAATTGAGATCAAATTTAGAACAAAAGAGCTGCTTGAGTCCATGGGTAGGCTTGATAAACAGTTGATCACATTGAAGGCAAAACTTCAAGAAGCTAGGAATAGCTGGAACTTTGGGATGGTTGAAGACTTACAACAGCAGATAAAATCTCGTGAAAAGCAACTTTTGCCCATATACACTCAAATAGCCACCAGATTTGCGGAGCTTCATGATTCTTCCCTAAGGATGGCTGCAAAGGGGGTGATCAGAGAAATTGTAGACTGGGATAAATCCCGTGCTTACTTCTATAAAAGGCTACGTAGGAGAATCGCTGAGGGTTCACTGATCAAGACTGTGAAAGATGCAGCTGGTGACCAGTTGTCCCATAAATCTGCAATGGACTTGATCAAAAACTGGTTTTTAGATTCTGATATTGCAAGAGGCAAAGAAGATGCTTGGGGGAATGATGAAGCTTTCTTTGCATGGAAGGATGATCAAGGGAAATATGAAGAAAAACTACAAGAGCTACGGGTTCAGAAAGTGTTGGTACAACTGACAAACATTGGTGACTCCATGTCAGATTTGAAAGCTCTACCTCAAGGTCTTGCTAACCATGGGGGCCGGGAGCATGCGACGTCGGGCCCATTCT

SEQ ID NO: 15 - DNA, 597 bp, J. curcas
gaggtgtatagctttcagatgcaacttggtggacccatagttatggcaacccatggagtagttcatcttactgtctcagatgatctagaaggtgtatctgctatcttgaactggctaagttgtatccctccttgtattggtggcacacttccaattttaggtccttcggatcctactgaaaggcctgtggagtatttcccagaaaactcatgtgatccacgtgctgctatttctggttctttggatggtaatgggaagtggcttgggggcatttttgacaagaatagttttgttgagacactggaaggctgggcaaggacagttgtgacaggaagggcaaagctcggaggaatccctgttggagtaatagctgttgaaactcaaactgtgatgcaggtgattcctgctgacccaggacagctcgattctcatgagagggttgttcctcaggctggccaagtatggtttccagattctgcaaccaaaacagctcaagctatattggatttcaacagagaagaacttccacttttcattcttgctaattggaggggcttttcaggtggacaaagggatctgttcgaaggaatccttcag

SEQ ID NO: 16 - DNA, 597 bp, J. curcas
ctgaaggattccttcgaacagatccctttgtccacctgaaaagcccctccaattagcaagaatgaaaagtggaagttcttctctgttgaaatccaatatagcttgagctgttttggttgcagaatctggaaaccatacttggccagcctgaggaacaaccctctcatgagaatcgagctgtcctgggtcagcaggaatcacctgcatcacagtttgagtttcaacagctattactccaacagggattcctccgagctttgcccttcctgtcacaactgtccttgcccagccttccagtgtctcaacaaaactattcttgtcaaaaatgcccccaagccacttcccattaccatccaaagaaccagaaatagcagcacgtggatcacatgagttttctgggaaatactccacaggcctttcagtaggatccgaaggacctaaaattggaagtgtgccaccaatacaaggagggatacaacttagccagttcaagatagcagatacaccttctagatcatctgagacagtaagatgaactactccatgggttgccataactatgggtccaccaagttgcatctgaaagctatacacctc

SEQ ID NO: 17 - protein, 508 aa, J. curcas
PIHSILIANNGMAAVKFMRSIRTWAYETFGNEKAILLVAMATPEDMKINAEHIRIADQFVEVPGGTNNNNYANVQLILEMAEGTRVDAVWPGWGHASENPELPDALSAKGIVFLGPPATSMAALGDKIGSSLIAQAADVPTLPWSGSHVKIPPESCLIAIPDEVYREACVYTTEEAIASCQVVGYPAMIKASWGGGGKGIRKVHNDDEVRALFKQVQGEVPGSPIFIMKVASQSRHLEVQLLCDQHGNVAALHSRDCSVQRRHQKIIEEGPITVAPLETVKKLEQAARRLAKSVNYVGAATVEYLYSMETGEYYFLELNPRLQVEHPVTEWIAEVNLPAAQVAVGMGIPLWQIPEIRRFYGVENGGGYDAWRKTSVVATPFDFDKAESTRPKGHCVAVRVTSEDPDDGFKPTSGKVQELSFKSKPNVWAYFSVKSGGGIHEFSDSQFGHVFAFGESRALAIANMVLGLKEIQIRGEIRTNVDYSIDLLHASDYRDNKIHTGWLDSRIA

SEQ ID NO: 18 - protein, 195 aa, J. curcas
ESCLIAIPDEVYREACVYTTEEAIASCQVVGYPAMIKASWGGGGKGIRKVHNDDEVRALFKQVQGEVPGSPIFIMKVASQSRHLEVQLLCDQHGNVAALHSRDCSVQRRHQKIIEEGPITVAPLETVKKLEQAARRLAKSVNYVGAATVEYLYSMETGEYYFLELNPRLQVEHPVTEWIAEVNLPAAQVAVGMGI

SEQ ID NO: 19 - protein, 75 aa, J. curcas
LLQNDHDPSKLVAETPCKLLRFLVLDGSHIEADTPYAEVEVMKMCMPLLSPASGVLQFKMSEGQAMQAGELIARL

SEQ ID NO: 20 - protein, 182 aa, J. curcas
FFYAVTDLACTKKLPLIYLAANSGARIGVAEEVKSCFKVGWSDETSPEGGFQYVYLSPEDYTHIASSVIAHELKLSNGETRWVIDDAIVGKEDGLGVENLSGSGAIASAYSRAYKETFTLTYVTGRTVGIGAYLARLGMRCMQRVVDQPIILTGFSALNKLLGREVYSSHIQLGGPKVMATN

SEQ ID NO: 21 - protein, 316 aa, J. curcas
GVVHLTVSDDLEGVSAILNWLSCIPPCIGGTLPILGPSDPTERPVEYFPENSCDPRAAISGSLDGNGKWLGGIFDKNSFVETLEGWARTVVTGRAKLGGIPVGVIAVETQTVMQQVIPADPGQLDSHERVVPQAGQVWFPDSATKTAQAILDFNREELPLFILAYWRGFSGGQRRDLFEGILQAGSTIVENLRTYNQPVFVYIPMMGELRGGAWVVVDSQINSDHIEMYADRTAKGNVLEPEGIIEIKFRTKELLESMGRLDKQLITLKAKLQEARNSWNFGMVEDLQQQIKSREKQLLPIYTQIATRFAELHDSS

SEQ ID NO: 22 - DNA, 29 bp, J. curcas
aatggaatggcrgcwgtsaagtttatacg

SEQ ID NO: 23 - DNA, 23 bp, J. curcas
catgtttttgcdtttggrgaatc

SEQ ID NO: 24 - DNA, 23 bp, J. curcas
ggcatgcacatcttcatracctc

SEQ ID NO: 25 - DNA, 22 bp, J. curcas
gaggtstayagcttycasatgc

SEQ ID NO: 26 - DNA, 21 bp, J. curcas
ctgaagdattccttcraavag

SEQ ID NO: 27 - DNA, 20 bp, J. curcas
ctcatytgrttgttgatscc

SEQ ID NO: 28 - DNA, 24 bp, J. curcas
agcaagwccttgwggyagagcttg

SEQ ID NO: 29 - DNA, 26 bp, J. curcas
gaggtbtayagctctcagatgcaact

SEQ ID NO: 30 - DNA, 26 bp, J. curcas
gtctcagatgatctagaaggtgtatc

SEQ ID NO: 31 - DNA, 23 bp, J. curcas
gtggacccatagttatggcaacc

SEQ ID NO: 32 - DNA, 24 bp, J. curcas
agaaagcttcatcattcccccaag

SEQ ID NO: 33 - DNA, 27 bp, J. curcas
actcctcaaggctataagaaccttaca

SEQ ID NO: 34 - DNA, 25 bp, J. curcas
cttggggtatggcacaagatcatct

SEQ ID NO: 35 - DNA, 24 bp, J. curcas
tgtgacaagtgaggatccagatga

SEQ ID NO: 36 - DNA, 27 bp, J. curcas
tggatgttatctctgaacaattcttca

SEQ ID NO: 37 - DNA, 23 bp, J. curcas
tggcaatgagaaggcaatcttgt

SEQ ID NO: 38 - DNA, 27 bp, J. curcas
tgaaaattcatgaatgcctccaccaga

SEQ ID NO: 39 - DNA, 25 bp, J. curcas
ggaacatacagtacactccatcacg

SEQ ID NO: 40 - DNA, 26 bp, J. curcas
agcaagatcggttacagcatagaaga

SEQ ID NO: 41 - DNA, 23 bp, J. curcas
agctgggctcaatgacattggca

SEQ ID NO: 42 - DNA, 20 bp, J. curcas
gatgcttgggggaatgatga

SEQ ID NO: 43 - DNA, 20 bp, J. curcas
acagccatggatggcatagg

SEQ ID NO: 44 - DNA, 21 bp, J. curcas
gtagggacatctgctgcttga

SEQ ID NO: 45 - DNA, 24 bp, J. curcas
gcttgagcaatcaaagatgagccg

SEQ ID NO: 46 - DNA, 21 bp, J. curcas
agccgattttatcacccagtg

SEQ ID NO: 47 - DNA, 15 bp, J. curcas
  misc_feature (1)..(1): n is a, c, g, or t
ntcgastwtsgwgtt

SEQ ID NO: 48 - DNA, 16 bp, J. curcas
  misc_feature (1)..(1): n is a, c, g, or t
ngtcgaswganawgaa

SEQ ID NO: 49 - DNA, 16 bp, J. curcas
  misc_feature (5)..(5): n is a, c, g, or t
wgtgnagwancanaga

SEQ ID NO: 50 - DNA, 16 bp, J. curcas
  misc_feature (4)..(4): n is a, c, g, or t
wgcnagtnagwanaag

SEQ ID NO: 51 - DNA, 16 bp, J. curcas
  misc_feature (6)..(6): n is a, c, g, or t
awgcangncwganata

SEQ ID NO: 52 - DNA, 28 bp, J. curcas
cgaagcttatggcttcctcagttctttc

SEQ ID NO: 53 - DNA, 26 bp, J. curcas
atcctaggcatgcattgcactcttcc

SEQ ID NO: 54 - DNA, 174 bp, J. curcas
atggcttcctcagttctttcctctgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatg

SEQ ID NO: 55 - DNA, 33 bp, J. curcas
atacctaggatgcagcatgttggaagcacaaag

SEQ ID NO: 56 - DNA, 27 bp, J. curcas
aacattctgggagctcgaaatcccttc

SEQ ID NO: 57 - DNA, 27 bp, J. curcas
gaagggatttcgagctcccagaatgtt

SEQ ID NO: 58 - DNA, 38 bp, J. curcas
ccgttaattaatcaactgatgacctttcgaagctcttc